(19) United States
(12) Patent Application Publication
(10) Pub. No.: US 2026/0275576 A1
(43) Pub. Date: Sep. 17, 2026

(54) ORTHOGONAL CLEAVAGE LIGATION SEQUENCING (OCLS)

(71) Applicant: Oregon Health & Science University, Portland, OR (US)

(72) Inventor: Corey M. Dambacher, Portland, OR (US)

(73) Assignee: Oregon Health & Science University, Portland, OR (US)

(21) Appl. No.: 19/469,255

(22) PCT Filed: Mar. 28, 2024
(86) PCT No.: PCT/US2024/022105

§ 371 Date: Sep. 25, 2025

Related U.S. Application Data
(60) Provisional application No. 63/492,777, filed on Mar. 28, 2023.

(51) Int. Cl. C40B 70/00; C12Q 1/6806; C40B 20/04

(52) U.S. Cl. CPC C40B 70/00; C12Q 1/6806; C40B 20/04

(57) ABSTRACT

The present disclosure relates to methods and systems involved with spatial encoding/decoding of features. Provided orthogonal cleavage-ligation sequencing (OCLS) systems encode two or more orthogonal recognition sites for restriction enzymes, such as Type IIS enzymes, for decoding of visual barcodes. Decoding employs specific orthogonal ligation of differentially-labeled probes, which enables visual distinguishing of the barcodes at each feature. This process can be repeated over cycles and in parallel.

### CROSS-REFERENCE TO RELATED APPLICATION

This is the 371 National Phase of International Application No. PCT/US2024/022105, filed Mar. 28, 2024, which claims priority to and the benefit of the earlier filing of U.S. Provisional Application No. 63/492,777, filed on Mar. 28, 2023, which is incorporated by reference herein in its entirety.

### INCORPORATION BY REFERENCE OF SEQUENCE LISTING

A computer readable file, entitled “0046-0082US_SeqList.xml” created on or about Sep. 24, 2025, with a file size of 28,672 bytes, contains the Sequence Listing for this application and is hereby incorporated by reference in its entirety.

### FIELD OF THE DISCLOSURE

The present disclosure relates generally to molecular barcodes, such as visual barcodes, and related methods and systems. More specifically, it relates to labeling compositions, methods, and workflows that overcome limitations of previously described visual barcoding.

### BACKGROUND OF THE DISCLOSURE

Determining the identity and/or location(s) of targets molecules (such as proteins or nucleic acids) in the sample can be vital for clinical applications, diagnostics, and biomedical research. In situ hybridization (ISH), immunohistochemistry, laser capture microdissection, and such technologies, enable visualization of the locations of target molecules within a sample, such as a biological sample.

The identities of target molecules also may be determined using methods (such as stochastic barcoding) that label target molecules, and track them through process of amplification and/or sequencing. However, there remains an on-going pursuit to develop new tools for improved spatial tracking, for their application toward single cell and spatial biology. Thus, there is a need for methods and systems that reliably correlate the identity of target molecule(s) with their location(s) within a sample, such as a substantially two-dimensional (2D) biological sample.

Next generation sequencing (NGS) technologies can be used for determining the sequence of barcoded beads that are coated with nucleic acids. However, traditional NGS sequencing requires expensive reagents for accurate determination of DNA sequences, which include reversibly-terminated and/or labeled nucleotides; an elevated temperature is required during decoding (usually 65° C.); multiple different reagents are introduced during each decoding cycle and these reagents do not function properly if they mixed (cleavage solution cannot be mixed with incorporation solution, for example), which requires employing sophisticated fluidics; and NGS cycle times take significantly longer than 5 minutes each. To read a large surface area of 1 μm beads via NGS, for example a 1.75 cm×1.75 cm surface of packed 1 μm beads where there will be about ten billion (10B) individual 1 μm beads, the length of the NGS locational barcode should be over 24 bp to reach a diversity of at least 200 T different beads in the library (potential locational barcode combinations) to reduce the chances of barcode redundancy within the large surface area. This results in requiring many more than six decoding cycles.

There remains a strong need in the art to develop additional methods for visual barcoding of targets, including in analysis of biological molecules.

### SUMMARY OF THE DISCLOSURE

Disclosed herein are methods and systems of visual barcoding that involve orthogonal cleavage-ligation sequencing (OCLS), as well as various components (including double stranded DNA oligonucleotides and hairpin-loop structure single stranded oligonucleotide) used in such methods and systems. Representative barcodes used for embodiments of OCLS as described herein are made of dsDNA. Visually decoded barcodes used in OCLS workflows are built over rounds of splitting and pooling (similar to that described in WO2022/187719), but the segments making up OCLS barcodes are not labeled. However, like the OCS method in WO2022/187719, each segment of the OCLS library is bound to a bead (or other solid support) or ligated to a previous segment to build a chain, and these segments can be co-encoded along with a NGS capture oligo (CO) barcode.

The current disclosure further provides methods of encoding and decoding (sequencing) of visual barcodes.

A method involving orthogonal cleavage-ligation sequencing (OCLS) of visual barcodes can be implemented to overcome issues associated with other sequencing methods, such as identimer chain decoding by orthogonal cleavage sequencing (OCS). This OCLS method is disclosed herein, including in FIGS. 1-6. Although OCS identimer chains may be composed of polymers other than nucleic acids for alternative decoding (such as peptide linkers using orthogonal proteases or chemical linkers using orthogonal chemical cleaving agents), the barcodes used for OCLS as described here must be comprised of double-stranded or partially double-stranded DNA (dsDNA). In brief, visually decoded barcodes used in OCLS workflows are also built over rounds of splitting and pooling, but the segments making up OCLS barcodes are not labeled (as they are with OCS identimers). Each segment of the OCLS library is ligated to a previous segment to build a chain, and these segments can be co-encoded along with a NGS capture barcode.

In similar fashion to splitting and pooling procedures outlined in WO 2022/187719, 100 or more different wells can be used in each round to build the OCLS bead libraries, over several rounds of splitting and pooling. For decoding, this method involves the use of at least one, but preferably two or more orthogonal REs, for instance Type IIS enzymes (examples of which are discussed and illustrated herein), in a single reaction mixture. This step is followed by specific orthogonal ligation of differentially-labeled probes, which enables visual distinguishing of the barcodes at each feature. This process is repeated over cycles as described herein, including in the accompanying Figures.

Provided herein are embodiments of visual barcodes including: at least two or more double-stranded DNA (dsDNA) oligonucleotide segments (cassettes) functionally linked linearly to each other, and each including within the sequence of the dsDNA segment: a recognition site (RS) for a specific restriction endonuclease (RE), a designed cleavage site (CS), or both a RS and a CS; and an untethered end at one end of the visual barcode. Optionally, the visual barcode is tethered to a solid substrate by a flexible linker attached at or near an end of the dsDNA of the visual barcode that is not the untethered end. Provided visual barcode embodiments do not include a visually detectable label. This is because the visually detectable label is provided by a probe used in conjunction with the barcode, as described herein.

In various visual barcode embodiments, the RE is a Type IIS restriction endonuclease, and the CS does not overlap the corresponding RS. Optionally, in a given visual barcode embodiment, at least one of the dsDNA segments includes a designed CS and the visual barcode includes a RS specific for a Type IIS RE, positioned appropriately such that the cognate Type IIS RE can cut the designed CS based on its position relative to the RS.

Also provided are collections of visual barcodes, wherein the collection includes a plurality of visual barcodes each of which includes a different set of dsDNA segments having different recognition sites (RSs) for specific restriction endonucleases (REs), designed cleavage sites (CSs), or both. In examples of collections of visual barcodes, at least two of the different visual barcodes are tethered to the same solid substrate.

Another embodiment is an orthogonal cleavage-ligation sequencing (OCLS) barcode including two or more dsDNA segments (cassettes), each dsDNA segment containing a recognition site (RS) for a specific restriction endonuclease (RE), and one or more overlapping region(s) configured to permit ligation to flanking dsDNA segments to form a chain of segments, which chain of segments constitutes the OCLS barcode.

Yet another embodiment is a visually detectable orthogonal ligation probe including: a fully or partially double-stranded DNA oligonucleotide, having a 3′ or 5′ overhang of at least two nucleotides, the sequence of which includes: a recognition site (RS) for a specific restriction endonuclease (RE), a designed cleavage site (CS), or both a RS and a CS; and covalently attached to the fully or partially dsDNA oligonucleotide, a visually detectable label (which optionally may be attached to the oligonucleotide by way of a flexible linker). In examples of the visually detectable orthogonal ligation probe embodiments, the fully or partially double-stranded DNA oligonucleotide includes: a linear double-stranded DNA oligonucleotide having a 5′ overhang; a linear double-stranded DNA oligonucleotide having a 3′ overhang; a hairpin stem-loop configured single-stranded DNA oligonucleotide having a 5′ overhang; or a hairpin stem-loop configured single-stranded DNA oligonucleotide having a 3′ overhang.

In further examples of the provided visually detectable orthogonal ligation probe, the visually detectable label includes one or more of a fluorescent label, a bioluminescent label, a chemiluminescent label, a chromophore, a quantum dot, a Raman label, a biotin moiety, or a radioactive isotope.

Optionally, the RE in the visually detectable orthogonal ligation probe is a Type IIS restriction endonuclease, and RS is specific for that Type IIS RE. By way of further example, the visually detectable orthogonal ligation probe can include a RS specific for a Type IIS RE, positioned appropriately so the cognate Type IIS RE can cut a designed CS based on its position relative to the RS.

Another embodiment is a collection of visually detectable orthogonal ligation probes as provided in any one of the probe embodiments, wherein the collection includes a plurality of visually detectable orthogonal ligation probes each of which includes a different recognition site (RSs) for specific restriction endonucleases (REs), or both a different RS and a different cleavage site (CS). Optionally, at least two of the different detectable orthogonal ligation probes in such collections include visually distinguishable detectable labels.

Also contemplated are collections of visually detectable orthogonal ligation probes, which include a plurality of different probes each of which is configured such that cleavage of that probe with the RE produces an overhang having a sequence different from at least 5, at least 7, at least 10, at least 12, at least 15, or more than 15 other probes in the collection. These collections of unique (within the set) probes can be used to “read” out results, based on detection of different visual signals that are dependent on the specific complementarity of sequence between a probe and the visual barcode to which it binds though their respective overhangs.

Yet another embodiment is an orthogonal cleavage-ligation sequencing (OCLS) oligonucleotide pair, including: a visual barcode, including: at least two or more double-stranded DNA (dsDNA) oligonucleotide segments (cassettes) functionally linked linearly to each other, and each including within the sequence of the dsDNA segment: a recognition site (RS) for a specific restriction endonuclease (RE), a designed cleavage site (CS), or both a RS and a CS; and an untethered end at one end of the visual barcode; and a visually detectable orthogonal ligation probe, including: a fully or partially double-stranded DNA oligonucleotide, having a 3′ or 5′ overhang of at least two nucleotides, the sequence of which includes: a recognition site (RS) for a specific restriction endonuclease (RE), or both a RS and a cleavage site (CS); and covalently attached to the fully or partially dsDNA oligonucleotide, a visually detectable label; wherein cleavage of the RS or the CS in the visual barcode produces a single-stranded “sticky end” overhang having a sequence with full complementary to a sticky end (overhang) produced by cleavage of the RS in the visually detectable orthogonal ligation probe.

Also provided are sets of OCLS oligonucleotides pairs, wherein each pair of visual barcode and visually detectable orthogonal ligation probe have a different fully complementary sequence overlap, and each visually detectable orthogonal ligation probe includes a different visually distinguishable detectable label.

Yet another embodiment is a method of encoding a visual barcode, the method including: contacting a double-stranded DNA (dsDNA) oligonucleotide tethered at a first end to a solid support, which dsDNA oligonucleotide has a single-stranded overhang at a second, untethered end, with a first dsDNA segment having a first overhanging end compatible for binding to the single-stranded overhang of the tethered dsDNA oligonucleotide and a second overhanging end, and including within the sequence of the first dsDNA segment: a recognition site (RS) for a specific restriction endonuclease (RE), a designed cleavage site (CS), or both a RS and a CS; which contacting occurs under conditions sufficient to allow specific hybridization of the single-stranded overhang of the first dsDNA segment to the single-stranded overhang of the tethered dsDNA oligonucleotide; ligating the first dsDNA segment to the tethered dsDNA oligonucleotide, to form a first captured barcode segment, which includes the second overhanging end of the first dsDNA segment; contacting the first captured barcode segment with a second dsDNA segment having a first overhanging end compatible for binding to the single-stranded overhang of the first captured barcode segment oligonucleotide and a second overhanging end, and including within the sequence of the second dsDNA segment: a RS for a specific RE different from the RS/RE in the first dsDNA segment, a designed CS different from the designed CS in the first dsDNA segment, or both; and ligating the second dsDNA segment to the first captured barcode segment, to form a second captured barcode segment, which includes the second overhanging end of the second dsDNA segment, which first captured barcode segment and second captured barcode segment constitute the visual barcode.

In examples of such method of encoding a visual barcode embodiment, the method further includes repeating the contacting and ligating steps one or more additional times, each time attaching an additional dsDNA segment to the captured barcode segments, to form the visual barcode. Optionally, in any method of encoding a visual barcode embodiment, at least one of the dsDNA segments includes a designed CS and the visual barcode includes a RS specific for a Type IIS RE, positioned appropriately so the cognate RE can cut the designed CS based on its position relative to the RS.

Also contemplated are examples of the methods of encoding a visual barcode, wherein visual barcodes are built using rounds of splitting and pooling using unlabeled DNA segments. By way of example, one or more rounds of splitting and pooling include(s): ligating one barcode cassette at a time onto a bead; splitting the resultant beads into individual compartments, optionally wells of a plate; ligating a different first compartment-specific barcode cassette onto the beads in each individual compartment, to yield a collection of beads containing different pairs of two barcode cassettes; washing the beads containing two barcode cassettes; pooling the beads containing two barcode cassettes; splitting the pooled beads containing two barcode cassettes into individual compartments, optionally wells of a plate; and repeating the ligating, washings, pooling, and splitting steps to increase diversity of the set of barcodes.

In any of the method of encoding a visual barcode embodiments, optionally the visual barcode includes a contiguous chain of dsDNA segments (cassettes), or the visual barcode includes at least two separate cassettes attached directly and separately to the solid support.

In any of the method of encoding a visual barcode embodiments, optionally the segments (cassettes) are co-encoded along with a next-generation-sequence (NGS) capture barcode.

Visual barcodes made by any of the provided methods are also encompassed within the disclosure.

Yet another embodiment is a method of decoding a visual barcode, including: contacting, in a milieu, at least one double-stranded DNA (dsDNA) orthogonal cleavage-ligation sequencing (OCLS) barcode including at least one restriction site (RS), with a restriction endonuclease (RE) that recognizes that RS, under conditions sufficient for the RE to cleave the dsDNA OCLS barcode, which RS/RE cleavage results in a single-stranded overhang to produce a partially single-stranded (ss) DNA-partially dsDNA OCLS barcode; contacting the partially ssDNA-partially dsDNA OCLS barcode with at least one orthogonal ligation probe including a dsDNA oligonucleotide including an overhang at a first end and a visually detectable label, under conditions sufficient for the overhang of the orthogonal ligation probe to bind by base pair-mediated hydrogen bonding to the overhang on the partially ssDNA-partially dsDNA OCLS barcode if the sequence of the overhang of the orthogonal ligation probe is the reverse complement of the sequence of the overhang of the partially ssDNA-partially dsDNA OCLS barcode; if base pair-mediated binding occurs, ligating the orthogonal ligation probe to the partially single-stranded (ss) DNA-partially dsDNA OCLS barcode to produce a captured probe; and detecting presence, absence, and/or quantity of captured probe by imaging the visually detectable label.

Optionally, in such a method embodiment the method may further include repeating the contacting/cleavage, contacting/base pair-mediated binding, ligating, and detecting steps cycle one or more times, where each additional cycle involves a cleavage of the dsDNA OCLS barcode at a different cleavage site (CS), base pair-mediated binding of a different orthogonal ligation probe, and/or detection of the presence, absence, and/or quantity of a different labeled captured probe.

In additional examples of the method of decoding a visual barcode embodiments, one or more of: the dsDNA OCLS barcode is attached to a bead or other solid surface; a plurality of different dsDNA OCLS barcodes are attached to a single bead or single address on another solid surface; the dsDNA OCLS barcode includes more than one non-overlapping RSs; at least one RS in the dsDNA OCLS barcode is recognized by a Type IIS RE, and cleavage occurs at a predetermined location outside of the RS; at least one RS in the dsDNA OCLS barcode is recognized by a RE that cleaves within the RS; or the visually detectable label includes at least one of a fluorescent label, a bioluminescent label, a chemiluminescent label, a chromophore, a quantum dot, a Raman label, or a radioactive isotope.

In examples of the method of decoding a visual barcode, the orthogonal ligation probe includes a single stranded DNA oligonucleotide having a stem-loop hairpin structure, wherein the overhang is at the end of the stem of the hairpin. For instance, optionally, after ligating, the method may further include contacting the milieu including the captured probe with a 5′-exonuclease.

Also provided are examples of the method of decoding a visual barcode, which include one or more of: contacting the at least one dsDNA OCLS barcode sequentially with two or more REs that each recognize a different, non-overlapping RS within the dsDNA OCLS under conditions sufficient for each RE to cleave the dsDNA OCLS barcode, each of which RS/RE cleavage results in a single-stranded overhang to produce a partially single-stranded (ss) DNA-partially dsDNA OCLS barcode; the ligating includes chemical ligation; or the ligating includes enzyme-mediated ligation.

Also provided are examples of the method of decoding a visual barcode, which include contacting two or more dsDNA OCLS barcodes, each including at least one RS, with a RE that recognizes that RS under conditions sufficient for the RE to cleave the dsDNA OCLS barcode, which RS/RE cleavage results in a single-stranded overhang to produce a partially single-stranded (ss) DNA-partially dsDNA OCLS barcode, wherein the RS/RE is different for each dsDNA OCLS barcode. For instance, such methods may include contacting the two or more dsDNA OCLS barcodes with two or more orthogonal REs. Optionally, the contacting with two or more orthogonal REs is simultaneous or sequential.

It is also contemplated in examples of the method of decoding a visual barcode are instances wherein following contacting with the one or more orthogonal REs, differentially-labeled probes are ligated using specific orthogonal reactions. For instance and further, the specific orthogonal ligation of differentially-labeled probes enables visual distinguishing of the barcodes at each feature in an array of visual barcodes.

Also provided are methods, wherein ligating the orthogonal ligation probe to the partially ssDNA-partially dsDNA OCLS barcode adds a new RS to the resultant a captured probe.

Also provided are methods of decoding visual barcodes using orthogonal cleavage-ligation sequencing (OCLS) essentially as described herein.

Yet another embodiment is an improved system for molecular barcoding, including repeated cycles of labeling, orthogonal cleavage, ligation, and imaging in order to identify individual features, wherein the orthogonal cleavage includes cleaving a double-stranded DNA barcode with a Type IIS Restriction Endonuclease. In examples of this system embodiment, two or more labeling and orthogonal probe ligation identification cycles occur in series, or occur concurrently.

Also provided are kits for carrying out one or more of the described methods, including kits that include one or more of the visual barcodes described herein (optionally attached to a bead or other solid support), one or more of the visually detectable orthogonal ligation probes described herein, and optionally other components useful in carrying out a method of encoding or decoding an OCLS barcode.

### BRIEF DESCRIPTION OF THE DRAWINGS

FIG. 1A illustrates a method for creating (encoding) OCLS barcodes, whereby each dsDNA segment used to construct the barcode contains a recognition site (RS) for a specific and different restriction endonuclease (RE), as well as one or more overlapping region(s) for ligation to flanking dsDNA segments to form a chain. Three rounds of barcode encoding via attachment of different segments to form a barcode chain are shown. The illustrated embodiment is carried out with the first dsDNA segment attached via flexible linker to a bead or other solid support. The illustrated method is described more fully herein.

FIG. 1B illustrates a method for creating OCLS barcodes, whereby each dsDNA segment used to construct the barcode contains a cassette including a RS designed to be a specific distance (number of bases) from cleavage site (CS) when the RS is recognized by a Type IIS RE (the designed cassette placing the RS the appropriate distance from the CS for the cognate Type IIS RE), as well as one or more overlapping region(s) for ligation to flanking dsDNA segments to form a chain. Three rounds of barcode encoding via attachment of different segments to form a barcode chain are shown. When creating OCLS barcode combinations containing designed cassette(s) having an engineered CS, the RS(s) optionally are included within the barcode chain. The illustrated embodiment reflects that the first dsDNA segment is attached via flexible linker to a bead or other solid support. The illustrated method is described more fully herein.

FIG. 2 illustrates two different OCLS barcode types. The barcode type shown on the left contains a RS for a RE that will cleave the OCLS barcode at the designed (engineered to be present) and corresponding RS. The barcode type shown on the right contains a RS for a Type IIS RE, which cleaves at a specifically-designed CS that is a set, predetermined distance from its RS (the distance being selected based on the RE being used). Methods of encoding and decoding these OCLS barcodes are described more fully herein.

FIGS. 3A-3B illustrate orthogonal ligation probes that can be used in OCLS decoding workflows. The dsDNA probes shown in the left side of FIG. 3A are formed by hybridization of two complementary strands of ssDNA or by the folding of a single ssDNA to form a hairpin with a double-stranded stem, and are labeled. The labels are illustrated as attached to the probe via a flexible linker. Probe embodiments include labeled dsDNA, or ssDNA in a hairpin structure as shown below the dsDNA probes in FIG. 3A and in FIG. 3B. The alternative embodiment shown in FIG. 3B provides probes that contain an encoded RS. The illustrated method is described more fully herein.

FIG. 4A illustrates a bead (or other solid support), to which is attached unlabeled barcodes, each having a different RS (RS1, RS2, RS3); the barcodes are orthogonally-cleaved with one or more restriction enzymes (REs), and the resultant product is imaged. FIG. 4B illustrates identifying orthogonally-cleaved barcodes step-wise using a first labeled, orthogonal ligation probe (identification (ID) cycle 1). FIG. 4C illustrates the second cycle of identifying orthogonally-cleaved barcodes step-wise using a second labeled, orthogonal ligation probe (ID cycle 2). FIG. 4D illustrates the third cycle of identifying orthogonally-cleaved barcodes step-wise using a third labeled, orthogonal ligation probes (ID cycle 3). FIG. 4E illustrates an option of identifying orthogonally-cleaved barcodes using more than one labeled, orthogonal ligation probe in a single ID cycle.

FIGS. 5A-5E illustrate an embodiment of orthogonal cleavage-ligation sequencing (OCLS), reflecting two different barcodes (of the type depicted in FIG. 1B and the right side of FIG. 2) each of which contains a different RS designed for recognition by a different Type IIS RE, as well as a designed cleavage site (CS) that is distanced by a precise number of bases from the RS depending upon the enzyme used. In FIG. 5A, two different Type IIS REs are added in a single, compatible reaction mixture to generate cleavage products 1 and 2 (aka, orthogonally cleaved products 1 and 2), which are removed in a subsequent wash step. An image is taken following cleavage by the enzymes to establish a baseline signal prior to decoding by orthogonal ligation with labeled probes. FIG. 5B shows both OCLS barcodes (top: stem1 oligo, bottom: stem2 oligo), with arrows pointing to both the RS and designed CS for each barcode oligo. For the first barcode oligo containing RS1 (top), the RS is positioned 10 bp upstream of the encoded CS. Following recognition at the RS element, cleavage by this enzyme (BsmFI) will occur at the designed CS element, exactly 10 bp downstream of the RS, which reveals a designed 5′-overhang of four bases in length. Similarly, for the second barcode oligo containing RS2 (bottom), the RS is positioned 14 bp upstream of the encoded CS. Following recognition at the RS element, cleavage by this enzyme (BpuEI) will occur at the designed CS element, exactly 14 bp downstream of the RS, which will reveal a designed 3′-overhang of two bases in length. FIG. 5C shows ligation of orthogonal labeled probes of the type shown in FIG. 3B, whereby probes contain an additional RS encoded within their dsDNA regions. Here, the encoded RS is specific for the same enzyme that cleaved the barcode in the prior cycle (though this is not required by the system). Probes shown in FIG. 5C also each contain a unique (within the reaction) detectable label, which corresponds to (that is, the label is indicative of) the specific overhanging bases the attached probe is designed to recognize (hybridize to) on cleaved OCLS barcode(s). Thus, the overhang on the probe and the overhang on the barcode match with correct base complementarity, and based on this association the barcoded feature can be identified upon imaging. as described elsewhere. Following ligation of a first series of probes, a wash step is performed followed by an imaging step to determine which label or combination of labels is present on each bead. As illustrated here (an example of one bead amongst a theoretical library of barcoded beads), a single label has been attached to the revealed sticky-end on the first OCLS barcode (stem1) via the compatible sticky end encoded within probe 1. Following the first imaging step, ligation of a second series of probes is performed in the same manner as the first, followed by an imaging step to determine which sticky ends were revealed following digestion of the second OCLS barcode (stem2). As shown in FIG. 5D, both the first series of ligation probes (top) and the second series of ligation probes (bottom) are orthogonal labeled probes of the type shown in FIG. 3B, which probes contain an additional RS encoded within their dsDNA regions. Due to the design illustrated in FIG. 5D, ligation of the probes creates a new CS either 10 bp or 14 bp for OCLS barcode 1 (top), and OCLS barcode 2 (bottom), respectively. Here, in both cases, the encoded RS contained within the labeled ligation probes is specific for the same enzyme that cleaved the barcode in the prior cycle (though this is illustrative rather than necessary). As shown in FIG. 5E, in the subsequent cycle of enzymatic digestion by these two Type IIS REs, a next designed sticky end (5′-overhanging stretch of 4 bases in length) will be revealed on OCLS barcode 1, and a next designed sticky end (3′-overhanging stretch of 2 bases in length) will be revealed on OCLS barcode 2. Following a wash step to remove cleavage products 1 and 2, these two barcodes would then be subjected to subsequent rounds of orthogonal ligation, starting with a series of labeled probes specific to sticky ends revealed by digestion with the first Type IIS enzyme (BsmFI), followed by an imaging step, followed by ligation with a series of labeled probes specific to sticky ends revealed by digestion with the second Type IIS enzyme (BpuEI). In FIG. 5E, the probes shown (probe 3 and probe 4) are orthogonal labeled probes of the type shown in FIG. 3B, whereby these probes contain an additional RS encoded within their dsDNA regions. Cycles of decoding may continue until all of the designed decoding sites of the OCLS barcodes are revealed and specifically detected, as described herein.

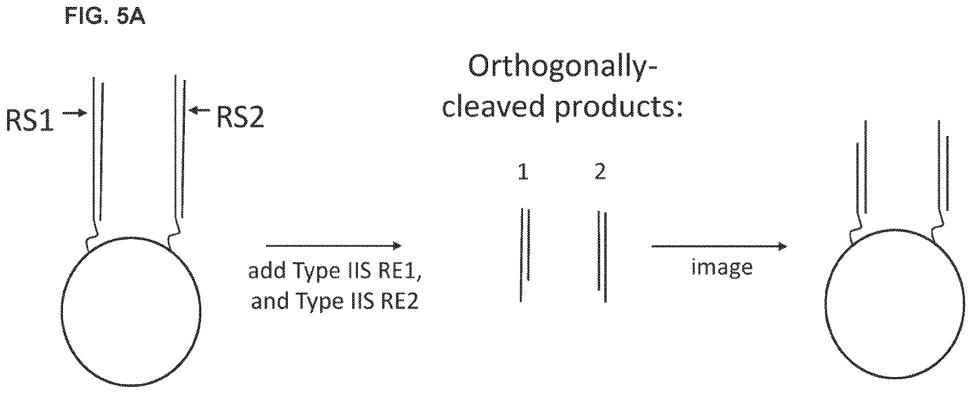

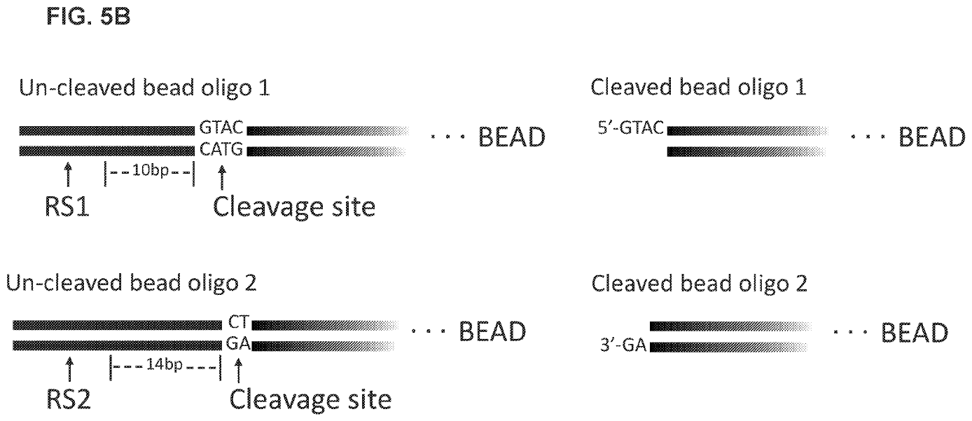

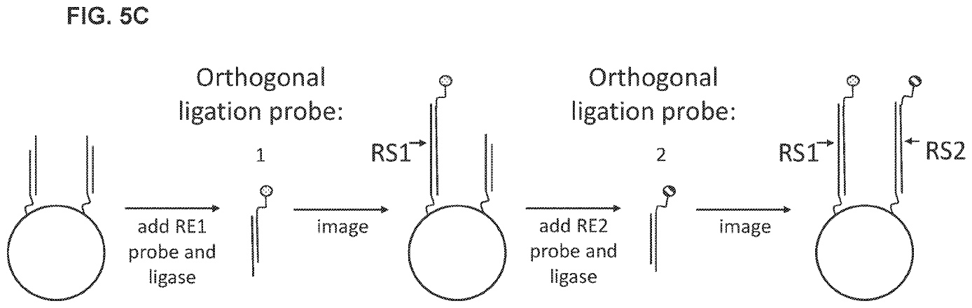

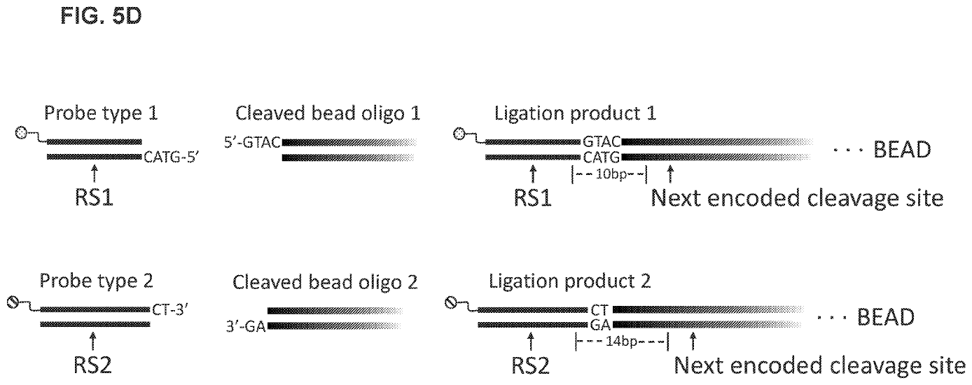

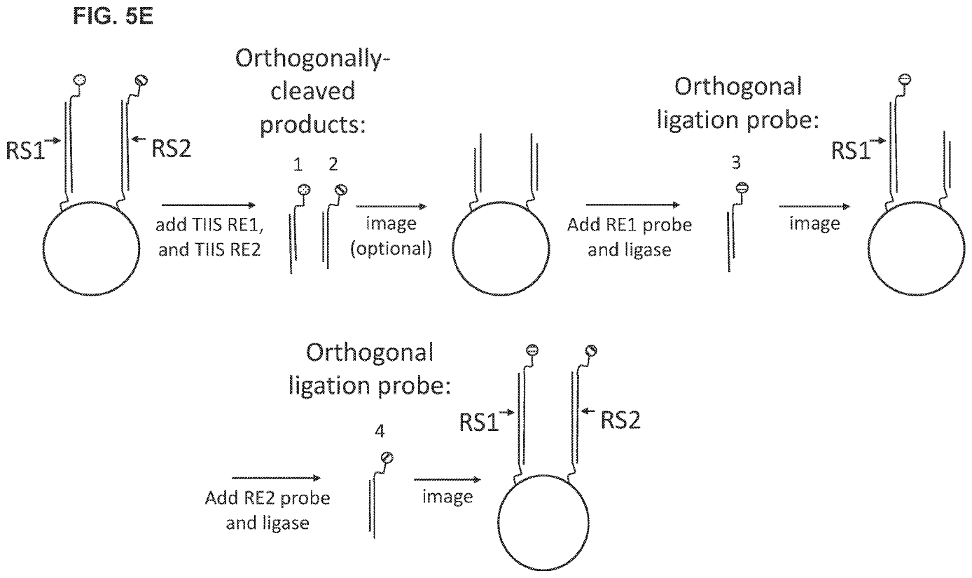

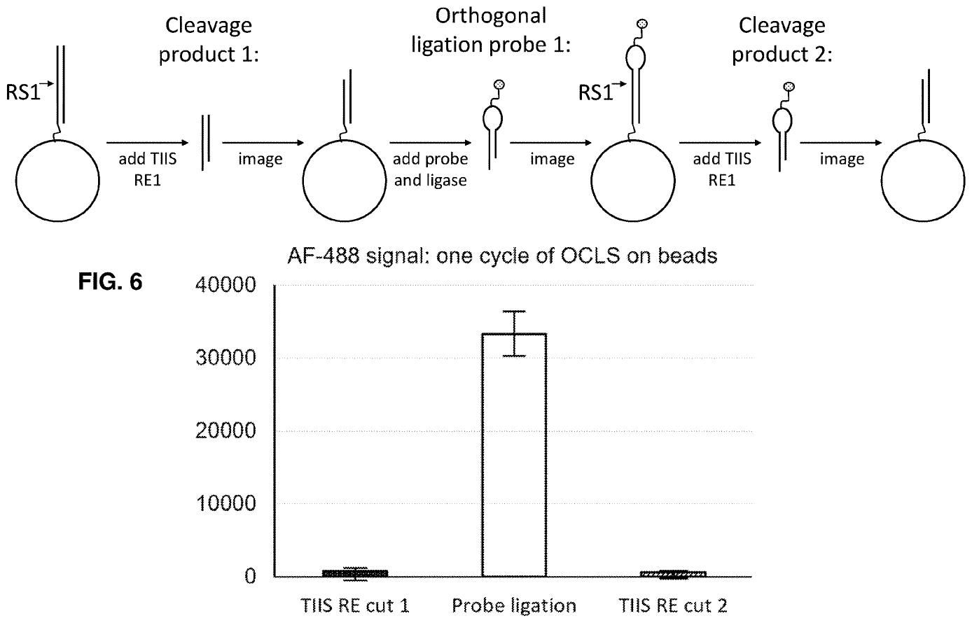

In summary, FIGS. 5A and 5B: Unlabeled barcodes orthogonally-cleaved with one or more Type IIS REs: step one. FIGS. 5C and 5D: Barcodes orthogonally-cleaved with one or more Type IIS REs, identified by orthogonal ligation: step two. This allows for “ratcheting” down the stem oligo toward the bead over cycles, with each cycle including the steps of: cleavage, ligation and imaging for decoding. FIG. 5E; Cleave, (ligate, image)×2: all subsequent cycles. Though illustrated with only two stem/RS pairs, this system can be operated (for instance, simultaneously) with several different stem/RS pairs. The two illustrated pairs have different overhangs following cleavage (one leaves a 5′ “sticky-end” for specific ligation, and the other leaves a 3′ “sticky-end” for ligation). The overhangs are of different lengths as well. Therefore, with this combination, the method guarantees there will not be incorrect ligation events (that is, there can be no cross-over between the two stems and ligation oligos). This figure illustrates an embodiment of the OCLS method involving multiplexing of (two) different “orthogonal” stem/RS pairs.

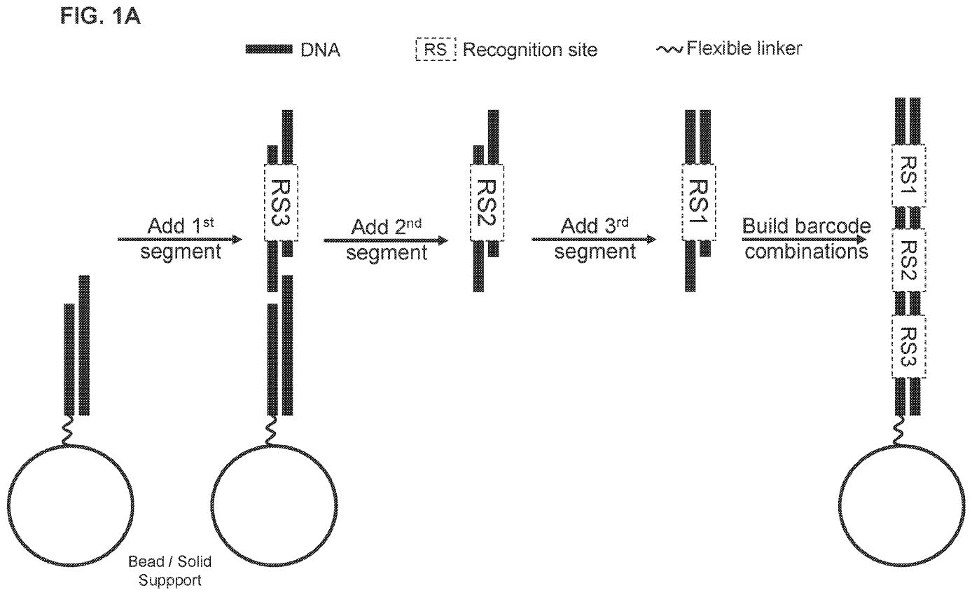

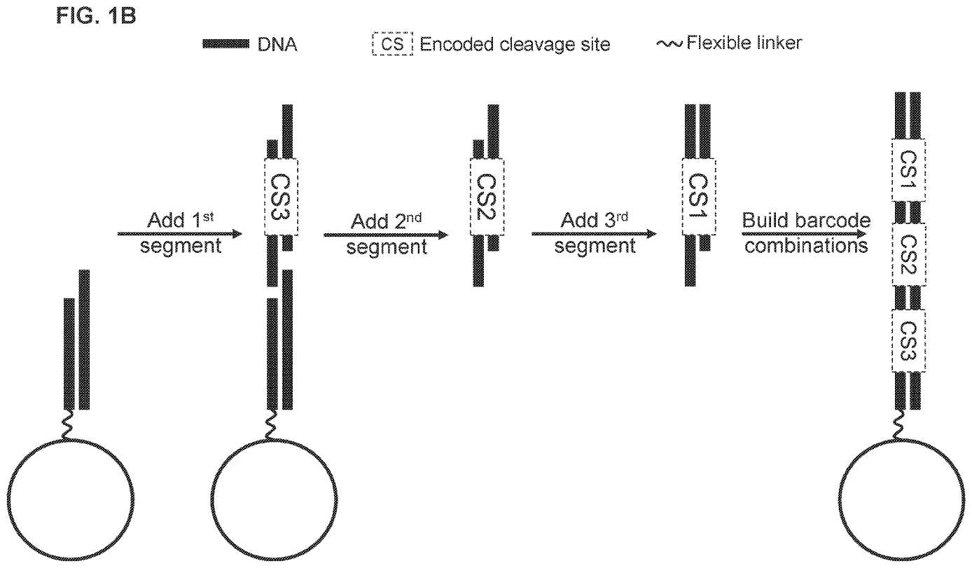

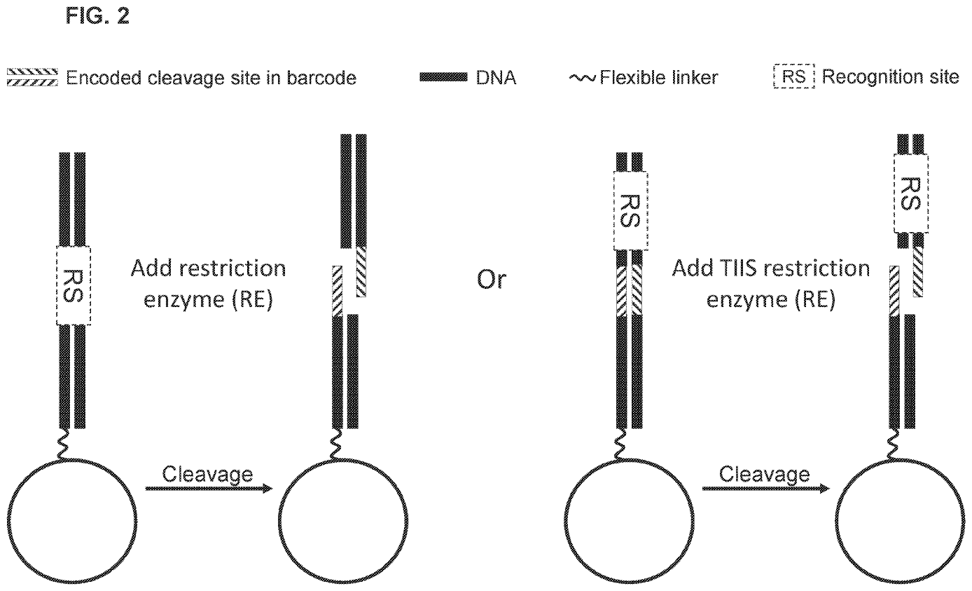

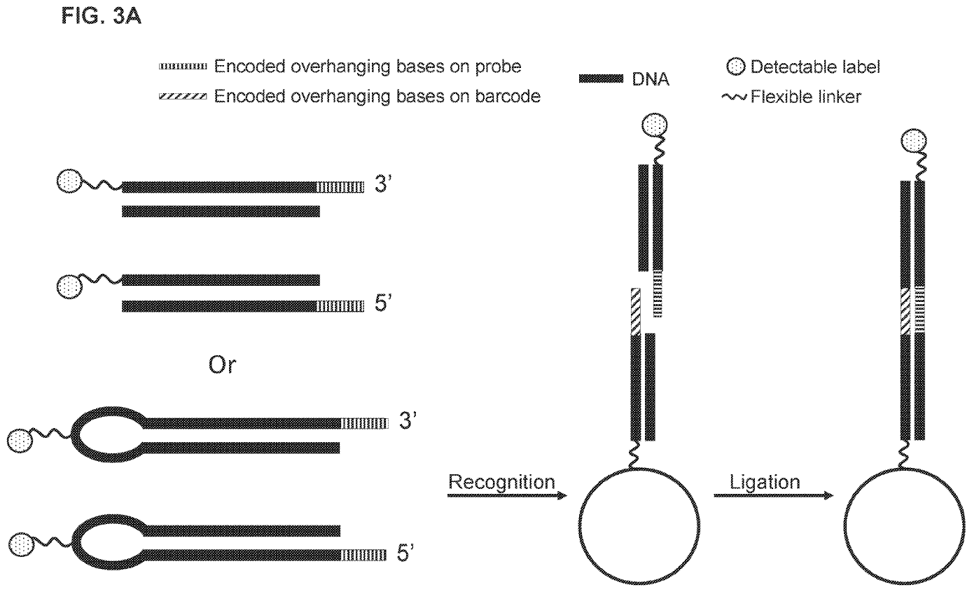

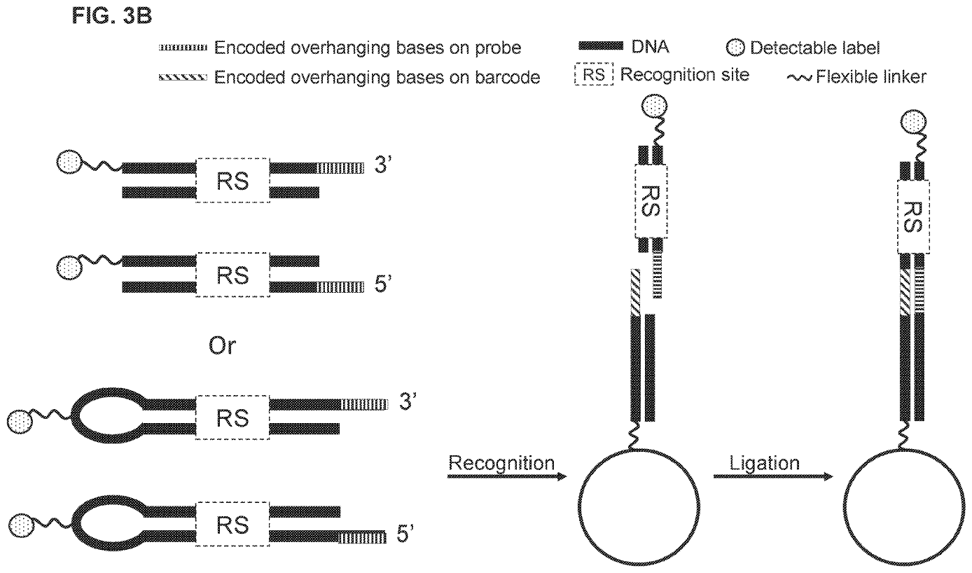

FIG. 6 shows an embodiment of the OCLS method and workflow, and a graph of the results from it as described in Example 1. A ssDNA hairpin probe of the type shown in FIG. 3B (bottom), labeled with AF-488 is demonstrated. The labeled ssDNA hairpin probe contains an encoded RS, and is used for orthogonal ligation following cleavage of an OCLS barcode of the type depicted in FIG. 1B (FIG. 2, right side) containing multiple encoded CS. For the experiment illustrated in FIG. 6, an imaging step was performed in the 488 nm (emission) channel to determine bead signal intensities following the first OCLS barcode cleavage by the Type IIS RE, BsmFI. Following probe ligation and a subsequent wash step, the beads were imaged in the 488 nm channel for determining signal obtained from the label, and therefore the revealed overhang on the cleaved OCLS barcode. The same RS for the Type IIS RE that was used in the first cleavage reaction (BsmFI) was also encoded in the ligated probe, so following a subsequent wash to remove components from the previous reaction, this enzyme was added to the beads to digest the OCLS barcode for a second time. Following the second digestion and a was step, an imaging step was performed in the 488 nm channel to determine signal obtained from the beads. Signal obtained across all three imaging steps in the 488 nm channel are plotted in the graph in FIG. 6, with relative fluorescence units (RFU) on the y-axis.

### REFERENCE TO SEQUENCES

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. Representative oligonucleotides that can be used with the OCLS workflow are shown below. Key: 5AmMC6=5′ flexible 6-carbon atom linker bearing a reactive primary amine; 5Phos=5′ phosphorylation, added so the oligo can be a substrate for T4 DNA ligase; iAmMC6T=internal 6-carbon atom amine-modified thymine.

SEQ ID NO: 1 is TIIS stem1 (BsmFI)
/5AmMC6/TTTTTTATTATATTACGCTAATAGCATTATATCATGTTAAC
GCAATGTCCCATATCGCTA
SEQ ID NO: 2 is TIIS stem1 comp
TAGCGATATGGGACATTGCGTTAACATGATATAATGCTATTAGCGTAAT
ATAAT
SEQ ID NO: 3 is TIIS s1hp1 (BsmFI)
/5Phos/CATGCGTCCCGCGTCGGATACGTTT/iAmMC6T/TTTCGTAT
CCGACGCGGGACG
SEQ ID NO: 4 is TIIS s1hp2 (BsmFI)
/5Phos/ATGCCGTCCCGCGTCGGATACGTTT/iAmMC6T/TTTCGTAT
CCGACGCGGGACG
SEQ ID NO: 5 is TIIS s1hp3 (BsmFI)
/5Phos/GCGTCGTCCCGCGTCGGATACGTTT/iAmMC6T/TTTCGTAT
CCGACGCGGGACG
SEQ ID NO: 6 is TIIS s1p1 (BsmFI)
/5AmMC6/TTTCGTATCCGACGCGGGACG
SEQ ID NO: 7 is TIIS s1p1 comp
/5Phos/CATGCGTCCCGCGTCGGATACGAAA
SEQ ID NO: 8 is TIIS s1p2 (BsmFI)
/5AmMC6/TTTCGTATCCGACGCGGGACG
SEQ ID NO: 9 is TIIS s1p2 comp
/5Phos/ATGCCGTCCCGCGTCGGATACGAAA
SEQ ID NO: 10 is TIIS s1p3 (BsmFI)
/5AmMC6/TTTCGTATCCGACGCGGGACG
SEQ ID NO: 11 is TIIS s1p3 comp c
/5Phos/GCGTCGTCCCGCGTCGGATACGAAA
SEQ ID NO: 12 is TIIS stem2 (BpuEl)
/5AmMC6/TTTTTTATTATATTAACTAATATGTATATAGTGTTAACAGT
CAATCTCAAGATATCGCTA
SEQ ID NO: 13 is TIIS stem2 comp
TAGCGATATCTTGAGATTGACTGTTAACACTATATACATATTAGTTAAT
ATAAT
SEQ ID NO: 14 is TIIS s2p1 (BpuEI)
/5AmMC6/TTTCGTATCCGACGCGCTTGAGGTACGACCT
SEQ ID NO: 15 is TIIS s2p1 comp
/5Phos/GTCGTACCTCAAGCGCGTCGGATACGAAA
SEQ ID NO: 16 is TIIS s2p2 (BpuEI)
/5AmMC6/TTTCGTATCCGACGCGCTTGAGGTACGACCA
SEQ ID NO: 17 is TIIS s2p2 comp
/5Phos/GTCGTACCTCAAGCGCGTCGGATACGAAA
SEQ ID NO: 18 is TIIS s2p3 (BpuEI)
/5AmMC6/TTTCGTATCCGACGCGCTTGAGGTACGACGT
SEQ ID NO: 19 is TIIS s2p3 comp
/5Phos/GTCGTACCTCAAGCGCGTCGGATACGAAA

### DETAILED DESCRIPTION

Efforts are under way to provide visual barcode systems that show improvements over traditional NGS, including reduced time, reduced cost of materials and reagents, no temperature cycling (that is, isothermal reactions) and lower overall operating temperature, and designing the system to allow all decoding cycles to occur in the same buffer (which enables simply adding each “next cleavage reagent” without removal of the previous one). These efforts have led to major improvements over NGS, including advances described as orthogonal cleavage sequencing (OCS) in WO 2022/187719. Following the decoding of OCS identimer chains on beads to determine bead locations on a surface, the NGS capture oligo (CO) containing the information regarding which identimer chain was on that bead (the color code) remains intact for subsequent capture of nucleic acids (the CO survives the decoding workflow described in WO 2022/187719 for example). The previously disclosed method of generating, encoding, and decoding visual barcodes relies on identimers and orthogonal cleavage sequencing (OCS), as described in WO2022/187719. In the previously disclosed method, beads containing identimers in combinations or in chains for use in OCS are combinatorially-encoded with visually detectable fluorophores/labels prior to the introduction of cleavage agents. OCS involves the introduction of a single cleavage agent at a time, followed by an imaging step in order to decode the sequences of identimer chains. In OCS workflows, the order of detectable labels within identimer chains are determined by a loss of signal following each cycle (as certain labels are removed from identimer chains during each cycle of orthogonal cleavage); and there is orthogonal cleavage but no ligation step required. Identimer chains (as used in OCS) may be composed of polymers other than nucleic acids for alternative decoding (such as peptide linkers using orthogonal proteases or chemical linkers using orthogonal chemical linker-cleaving agents).

Disclosed herein are methods involving orthogonal cleavage-ligation sequencing (OCLS) of visual barcodes that can be implemented to overcome various limitations associated with other sequencing methods, such as identimer chain decoding by orthogonal cleavage sequencing (OCS; see WO2022/187719). The barcodes used for OCLS as described herein, in some embodiments, are dsDNA. The double-stranded nature can arise from two single-stranded DNA molecules paired, or with a single-stranded DNA that folds back on itself to form a hairpin (where the stem of the hairpin is double-stranded). In brief, visually decoded barcodes used in OCLS workflows are built through rounds of splitting and pooling (analogous to that described in WO2022/187719; see also Rosenberg et al., Science 360 (6385): 176-182, 2018; Kuchina et al., Science 371 (6531): doi:10.1126/science.aba5257, 2020; U.S. Pat. No. 11,692,214; US Pat. Publication US20220403452A1), but the segments making up OCLS barcodes are not labeled (as they are with the identimers of WO2022/187719). Individual segments of an OCLS library are each ligated to a previous segment to build a chain; these segments can be co-encoded along with a NGS capture barcode.

In similar fashion to splitting and pooling procedures outlined in WO 2022/187719, 100 or more different wells can be used in each round to build the bead libraries, over several rounds of splitting and pooling. For decoding, this method involves using at least one, but optionally two or more orthogonal REs, for instance Type IIS enzymes (for example, those used in Figures and legends described herein), in a single reaction mixture. This step is followed by specific orthogonal ligation of differentially-labeled probes, which enables visual distinguishing of the barcodes at each feature, based on their encoded sticky-ends (overhanging bases). This process is repeated over cycles as described in the figures and legends.

Different barcodes that are compatible for use in orthogonal cleavage ligation sequencing (OCLS) workflows can be created by adding dsDNA segments containing encoded restriction endonuclease (RE) recognition sites (RS) and/or cleavage sites (CS), in different combinations to beads or other solid support features. The dsDNA OCLS barcode stem oligo shown in FIGS. 1A, 1B can be used for building such barcodes. Here, a dsDNA stem region is attached at one of its 5′-ends to a bead through a flexible (e.g., polyethylene glycol (PEG₁₂)) linker; in this illustration the stem dsDNA oligo is common to all beads contained within a library. At the opposing end, the dsDNA stem oligo contains a 5′ overhang or “sticky-end” (region of single stranded DNA bases) designed for ligation to the next barcode segment. Optionally, different stems (distinguishable by sequence content and/or “sticky-ends”) or different cleavable segments of OCLS barcodes can be attached at different locations on beads to increase diversity of encoding. In this way, many different individual segments or multiple chains of different content or type can be built in a variety of combinations using a splitting and pooling approach.

Though exemplified herein using enzymatic ligation, the use of chemical ligation to capture probes on OCLS barcodes is also contemplated.

FIG. 1A shows a method for creating OCLS barcodes, whereby each dsDNA segment contains a recognition site (RS) for a specific restriction endonuclease (RE), as well as one or more overlapping region(s) for ligation to flanking dsDNA segments to form a chain. The figure illustrates three rounds of barcode encoding via attachment of different segments to form a barcode chain. When creating OCLS barcode combinations containing RS for recognition and cleavage by a specific RE (the enzyme cleaves at the site of recognition), many different RS must be included in each round of combinatorial encoding. For example, two OCLS barcodes are decoded by ligation to differentially-labeled probes, so diversity of barcodes shown in FIG. 1A is limited by the number of different options for probe ligation (probes containing compatible sticky-ends for ligation to cleaved barcodes), and therefore each round of information encoding is directly proportional to the number of different RS used. During decoding of OCLS barcodes by orthogonal ligation, whereby each decoding probe (contained within a pool of probes) contains an overlapping region or “sticky-end” that is compatible for ligation with one or more of the cleaved barcode oligos, one uniquely-distinguishable label (or unique combination of distinguishable labels) attached to probes is used for determining which sticky-end is available/revealed on each bead. For each of the cleavable barcode segments within this OCLS barcode type (that is added during each round of encoding), each unique RS will correspond to only a single label or combination of labels during decoding: one orthogonal RS is determined by only one distinguishable label (or distinguishable combination of labels) during decoding.

FIG. 1B shows a method for creating OCLS barcodes, whereby each dsDNA segment contains a cleavage site (CS) that can be designed to be present in the sequence, as well as one or more overlapping region(s) for ligation to flanking dsDNA segments to form a chain. The figure illustrates three rounds of barcode encoding via attachment of different segments to form a barcode chain. When creating OCLS barcode combinations containing designed CS, inclusion of the RS within the barcode chain itself is optional but not necessary. That is because CS can be designed for cleavage by Type IIS REs, and these enzymes have recognition sites (RS) that are distant from the site at which the enzyme cleaves dsDNA. These enzymes will cleave any site that is a certain number of bases (specific for each enzyme) away from their RS, and they will leave a certain number of 5′- or 3′-overhanging bases, depending on the specific enzyme/RS used. Therefore, orthogonality of the barcode/probe pairs for this OCLS barcode type can be designed, which significantly increases the number of different ligation probes that can be used for each RS. When using Type IIS REs, the RS can be included on the outer-most segment, enabling recognition by the enzyme at the included RS, and dsDNA cleavage by the enzyme at a designed cleavage site (the location of which is picked based on the Type IIS RE and RS being used). Additionally, a Type IIS enzyme-specific RS can be included on (encoded within) the incoming labeled orthogonal ligation probes to enable a subsequent cleavage reaction by the same Type IIS RE or by a different Type IIS RE. This enables encoding of just the designed CS (along with necessary flanking sequences for building the barcodes) within this OCLS dsDNA barcode type.

In FIG. 2, two different OCLS barcode types are shown. These barcode types can be attached to beads at different sites around the bead to generate different combinations, or segments of these barcodes can be attached to form barcode chains as shown in FIGS. 1A, 1B, via a splitting and pooling approach. Following barcode construction, barcoded beads can be immobilized on a surface, for example in a flow cell, for decoding. The barcode type shown on the left contains a recognition site (RS) for a restriction enzyme (RE) that will cleave the OCLS barcode at the designed and corresponding RS. Following cleavage, any dsDNA (or ssDNA hairpin) downstream of the RS (from the bead attachment site) is removed during a wash step, revealing a sticky-end for compatible ligation to a specifically-labeled probe during decoding. For the OCLS barcode type shown on the left, multiple different RS (and therefore multiple different, orthogonal REs) are required for revealing multiple different sticky-ends for downstream decoding by orthogonal ligation with differentially-labeled probes. The barcode type shown on the right contains a RS for a Type IIS RE, which cleaves at a specifically-designed site that is distant from its RS. Following cleavage by the Type IIS RE, any dsDNA (or ssDNA hairpin) downstream of the encoded cleavage site is removed during a wash step, potentially revealing a series of differentially-designed sticky-ends capable of being recognized by one probe in a series of designed ligation probes.

FIG. 3A shows orthogonal ligation probes that can be used in OCLS decoding workflows. As outlined above, decoding of individual OCLS barcodes involves recognition by one designed probe in a series of probes, whereby each probe in the series of probes contains one or more distinguishable labels, and whereby each probe's distinguishable label (or associated, distinguishable combination of labels) corresponds to (contains bases that are complementary with) one of the differentially-designed sticky-ends revealed on the cleaved OCLS barcode(s). dsDNA probes shown in FIG. 3A are formed by hybridization of two complementary strands of ssDNA, and are labeled using NHS-modified fluorophore reagents for covalent attachment to the dsDNA through a flexible, 6-carbon linker on one end (bearing a 5′-amino modification). On the opposing end of the duplex, these probes contain a specific region of overhanging bases, in order to create a designed 5′- or 3′-overhang. The overhanging bases of the probes correspond to a known label or known combination of labels (attached to probes), such that during decoding (following a ligation step and an imaging step), detectable label(s) are used for determining the overhanging bases revealed on different OCLS barcodes. Probes can be comprised of labeled dsDNA DNA, or may be comprised of ssDNA in a hairpin structure as shown below the dsDNA probes in FIG. 3A and in FIG. 3B. Alternatively, as shown in FIG. 3B, probes may contain an encoded RS. The RS encoded within orthogonal ligation probes used for decoding OCLS barcodes may correspond to an RS that is recognized by a Type IIS RE. The RS may have been used in a previous cycle of decoding, or may be used in a future cycle of decoding. In this way, the same Type IIS RE site (RS) can be used in every cycle of decoding, enabling a ratcheting effect as OCLS barcodes are decoded. Alternatively, a different Type IIS RS can be encoded in probes that are used in each cycle of decoding, to reduce aberrant cleavage across cycles (in this case, any OCLS barcodes left un-cleaved in a previous cycle will not be recognized in a subsequent cycle). Likewise, the use of labeled hairpin probes during decoding creates labeled products (following ligation) that do not have an open 5′-phosphorylated end. In this case, all oligos that did not receive a hairpin ligation probe can be removed with the use of a 5′-exonuclease, such as lambda exonuclease, which recognizes only the 5′-phosphorylated end of oligos for digestion. By altering the Type IIS RS encoded within probes during each cycle of decoding, and by including a 5′-exonuclease clean-up step after ligation, any phasing (observed signal corresponding to an incorrect cycle) during OCLS decoding can be reduced.

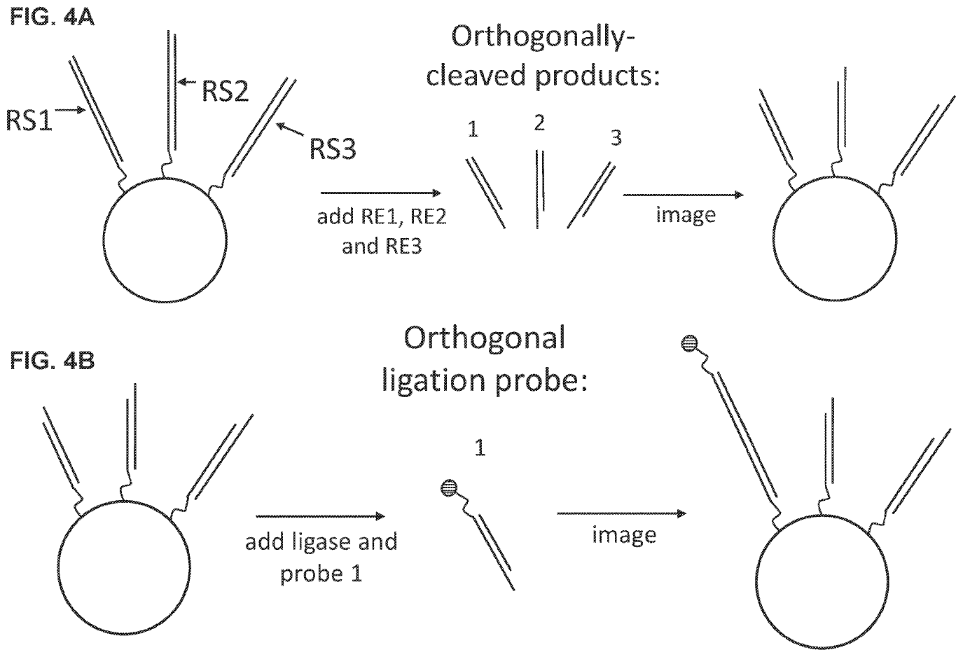

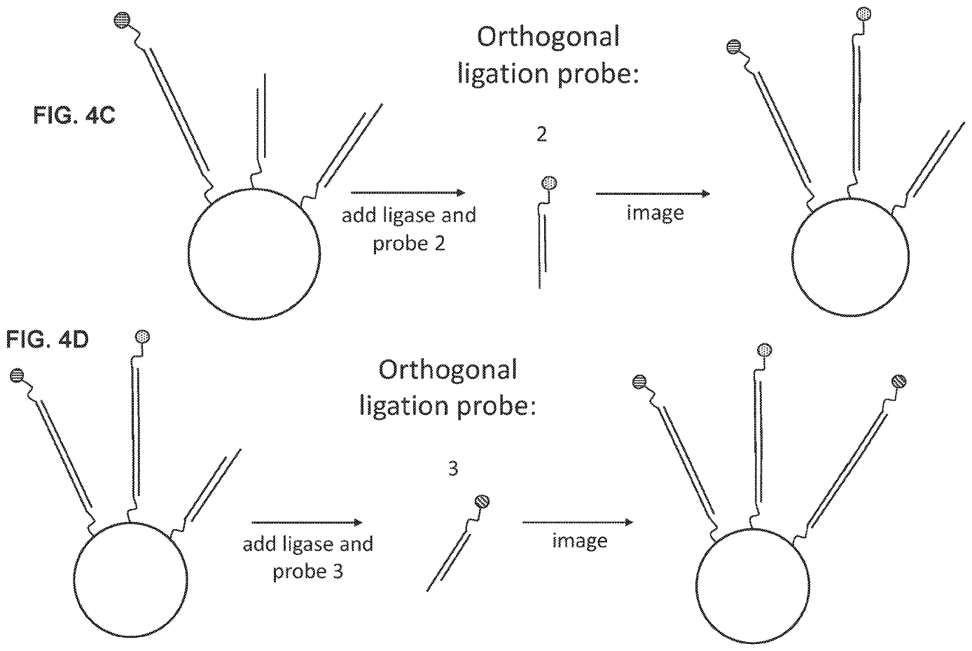

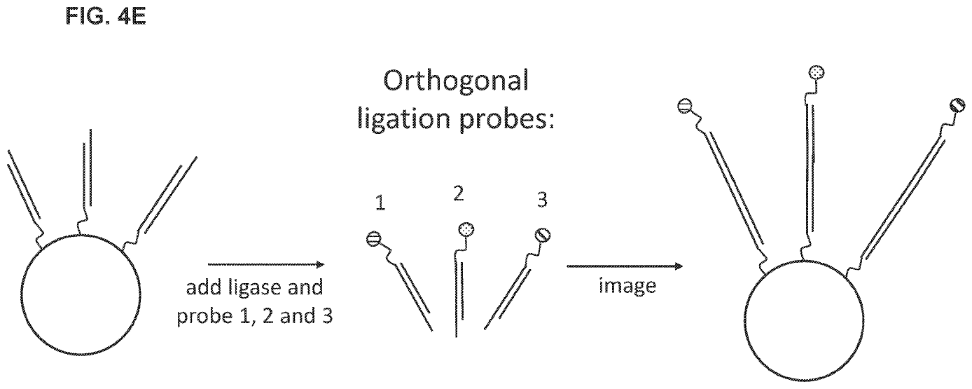

FIG. 4A depicts a bead containing three different segments of the OCLS barcode type shown in FIG. 1A (and FIG. 2, left side) attached at three different locations on the bead. Beads may be immobilized on a surface prior to decoding, for example, in a flow cell. Each segment contains a unique RS (RS1-RS3), which is recognized and cleaved by one of three different, specific, and orthogonal REs. These three enzymes can be used sequentially or can be included within a single reaction mixture containing a commonly-compatible buffer supplied by the manufacturer (e.g., NEB 1× CutSmart Buffer). Following digestion with all three enzymes, the three different cleavage events reveal overhanging bases of different compositions (orthogonal overhangs), whereby stems of each type can be detected by orthogonal ligation to differentially-labeled detection probes of the types shown in FIGS. 3A and 3B. FIG. 4B shows ligation of a first labeled probe to the cleaved OCLS barcode segment, which contains a compatible overhang for ligation with the first probe. The label on the first probe is used for determining which overhanging bases on the OCLS barcode were revealed by digestion and were then recognized by the probe for compatible ligation. In FIG. 4B, an image taken of the bead following ligation will show a single label or combination of labels corresponding to the detectable label(s) contained on probe 1. FIG. 4C shows ligation of a second labeled probe to the cleaved OCLS barcode segment, which contains a compatible overhang for ligation with the second probe. As with the first probe, the label on the second probe is used for determining which overhanging bases were revealed on the OCLS barcode following digestion with the enzymes. In FIG. 4C, an image taken of the bead following ligation of the second probe will show a single label or combination of labels corresponding to the detectable label(s) contained on both probe 1 and on probe 2. FIG. 4D shows ligation of a third labeled probe to the cleaved OCLS barcode segment, which contains a compatible overhang for ligation with the third probe. As with the first and second probes, the label on the third probe is used for determining which overhanging bases were revealed on the OCLS barcode following digestion with the enzymes. In FIG. 4D, an image taken of the bead following ligation of the third probe will show a single label or combination of labels corresponding to the detectable label(s) contained on probe 1, probe 2, and probe 3. In this example, a single cleavage event may contain multiple enzymes in a single reaction mixture, but the number of decoding cycles (ligation followed by imaging) is repeated as many times as necessary to decode the number of [revealed overhangs on] stems that are intended to be detected on the bead.

Alternatively, one or more REs may be used within a single reaction mixture as described above, but this step can be followed by the use of multiple different labeled probes contained within a pool (or series of probes) designed to create combinations of labels that are used for decoding OCLS barcodes. FIG. 4E shows the use of three different segments of the OCLS barcode type shown in FIG. 1A (and FIG. 2, left side), attached at three different locations on a bead. These three OCLS barcodes can then be digested (cleaved) by one or more REs to create combinations of sticky-ends for detection by corresponding probes during one or more cycles of OCLS for decoding.

FIGS. 5A-5E illustrate an embodiment of orthogonal cleavage-ligation sequencing (OCLS). The stem1 oligos (which pair with RS1) once hybridized, contain a formed dsDNA BsmFI recognition site (RS1); this Type IIS enzyme cleaves 10 bp downstream of its recognition site and leaves a four base, 5′ overhang. The stem2 oligos (which pair with RS2) once hybridized, contain a formed dsDNA BpuEI recognition site (RS2); this Type IIS enzyme cleaves 14 bp downstream of its recognition site and leaves a two base, 3′ overhang (FIGS. 5A and 5B; Unlabeled barcodes orthogonally-cleaved with one or more Type IIS REs: step one). These two enzymes will cut exactly this distance (respectively) downstream of their recognition sites every time they cleave DNA, but the site they cleave is not sequence-specific. This enables coordinated design of orthogonal cleavage sites (overhangs or “sticky ends”) with labeled orthogonal probes that contain compatible overhangs, such that each distinguishably-labeled probe recognizes a specifically-designed cleavage site. dsDNA barcodes (stems) containing defined 5′- or 3′-overhangs in different order (going from 5′ to 3′ for example) can be encoded by a splitting and pooling ligation strategy to generate libraries of dsDNA barcodes. The labeled ligation probes are covalently-attached to cleaved oligos by orthogonal ligation, and this newly formed ligation product contains a detectable label that is imaged for identification of the specific overhang sequence. The ligate-able probes also contain a dsDNA region encoding a Type IIS recognition site to enable subsequent cleavage at a different location on the stem oligo following ligation (FIGS. 5C and 5D; Barcodes orthogonally-cleaved with one or more Type IIS REs, identified by orthogonal ligation: step two). This allows for “ratcheting” down the stem oligo toward the bead over cycles, with each cycle containing the steps of: cleavage, ligation and imaging for decoding (FIG. 5E; OCLS preferred: Cleave, (ligate, image)×2: all subsequent cycles). In the case where more than one stem type is used in the same OCLS decoding workflow, cleavage is performed by more than one enzyme within a single reaction mixture, and a cycle of ligation and imaging is implemented for each stem type included. Labeled hybridized dsDNA ligation probes are used for both the BsmFI- and BpuEI-specific stems (stem1 and stem2 respectively), and labeled DNA hairpin ligation probes have also been designed for the BsmFI stem. Though illustrated with only two stem/RS pairs, this system can be operated (for instance, simultaneously) with several different stem/RS pairs. The two illustrated pairs have different overhangs following cleavage (one leaves a 5′ “sticky-end” for specific ligation, and the other leaves a 3′ “sticky-end” for ligation). The overhangs are of different lengths as well. Therefore, with this combination, incorrect ligation events are virtually eliminated (that is, there can be no cross-over between the two stems and ligation oligos). Thus, this figure illustrates a demonstration of the method involving multiplexing of (two) different “orthogonal” stem/RS pairs.

FIG. 6 shows a preferred embodiment of the OCLS method and workflow, and results from Example 1. Here, the use of a ssDNA hairpin probe of the type shown in FIG. 3B (bottom), labeled with AF-488 is demonstrated. The labeled ssDNA hairpin probe contains an encoded RS, and is used for orthogonal ligation following cleavage of an OCLS barcode of the type depicted in FIG. 1B (FIG. 2, right side) containing multiple encoded CS. For the experiment outlined in FIG. 6, an imaging step was performed in the 488 nm (emission) channel to determine bead signal intensities following the first OCLS barcode cleavage by the Type IIS RE, BsmFI. Following probe ligation and a subsequent wash step, the beads were imaged in the 488 nm channel for determining signal obtained from the label, and therefore the revealed overhang on the cleaved OCLS barcode. The same RS for the Type IIS RE that was used in the first cleavage reaction (BsmFI) was also encoded in the ligated probe, so following a subsequent wash to remove components from the previous reaction, this enzyme was added to the beads to digest the OCLS barcode for a second time. Following the second digestion and a was step, an imaging step was performed in the 488 nm channel to determine signal obtained from the beads. Signal obtained across all three imaging steps in the 488 nm channel are plotted in the graph in FIG. 6, with relative fluorescence units (RFU) on the y-axis. From these experiments it was clear that ligation of the probe and subsequent cleavage of the probe were successfully demonstrated. Following the first cleavage by BsmFI (TIIS RE cut 1), beads contained nearly the same mean intensity value (<1,000 RFU) as they did following the second cleavage by BsmFI (TIIS RE cut 2), but following ligation of the ssDNA hairpin probe labeled with AF-488, the mean intensity value of beads was about 33,000 RFU. Ligation reactions containing a labeled, incorrect ssDNA hairpin probe (hairpin probe 3, containing an incorrect overhang for the OCLS barcode) did not create a ligation product (also determined by gel analysis). This experiment shows the possibility that many different beads in a library that are encoded with different combinations of OCLS barcodes containing different CS can be decoded in highly parallel fashion by using the OCLS workflow outlined here. This method involves ratcheting down the OCLS barcode by adding labeled orthogonal ligation probes containing encoded RS for use with Type IIS REs. In a preferred embodiment, RS corresponding to Type IIS enzymes are used as described above, and individual probes used in each cycle of decoding are comprised of labeled, ssDNA hairpin oligos containing specific overhangs for recognition of revealed overhangs on cleaved OCLS barcodes as shown in FIG. 6. Preferably, a different Type IIS RS is included in each subsequent probe that is used in each subsequent cycle, to reduce aberrant cleavage across cycles (not shown here). As mentioned elsewhere, this approach will reduce the propensity for an enzyme that was used in a previous cycle to cleave an intact OCLS barcode that may have survived digestion (left un-cleaved) following a previous cleavage cycle. Additionally, the use of labeled ssDNA hairpin oligos as the ligation probes enables a clean-up step following the ligation step, which can be performed before or after the imaging step (as described previously with the use of a 5′-exonuclease such as lambda exonuclease, which recognizes 5′-phosphorylated ends for digestion and removal of that strand, making these barcodes incapable of interacting with any subsequent enzymes or probes used in future cycles). The clean-up step will remove any remaining dsDNA following a ligation step that was not 100% efficient, as ligated products (containing the labeled ssDNA hairpin oligos) can be designed such that they do not contain open 5′-phosphorylated ends. Taken together, the preferred embodiment outlining the use of ligation probes consisting of labeled ssDNA hairpins that contain a different RS in each cycle to reduce aberrant cleavage in subsequent cycles, combined with the described clean-up step to remove 5′-phosphorylated DNA performed following the ligation step, should enable a workflow capable of achieving multiple cycles of accurate OCLS barcode decoding.

The herein provided methods were created for multiplexing many differentially barcoded beads in a single experiment. In such uses, it is important need to determine which bead/location on a solid support is which amongst many beads/location; thus, each bead/location in a library must have a unique code (there can be many copies of each unique bead/location, but each individual addressable location in a library must have a unique code). Described herein are methods, components, and systems useful for determining which bead/location has which barcode using visual labels, which are ligated to specific sticky-ends which become revealed on barcodes following digestion by a restriction enzyme. Therefore, what is being read out in the described approaches are the specific sticky-ends on the barcodes using one unique label (or unique combination of labels) for each different sticky end that is revealed. This relies on:

(1) A cleavable barcode.
a) Because barcodes are being read out using labeled probes that must find their cognate match, the easiest way to do this is with dsDNA barcodes cleaved by enzymes, for which specific labeled probes can be designed that will find their match by base pairing and subsequent ligation. The reason for that is because it is something that can be performed in cycles; over many cycles it is possible to read barcodes of higher and higher diversity. If only a single cycle is desired, there are other ways to do it:
   i) This could also be accomplished using specific, orthogonal recognition by antibodies that are finding their cognate analytes, whereby antibodies are labeled and are therefore able to generate combinations (color codes) of labels on features by binding analytes in different combinations.
   ii) This could be accomplished using differentially-labeled DNA or RNA binding proteins, preferably using proteins that will recognize a specific DNA or RNA element amongst many different elements.
b) To multiplex many barcodes, orthogonality is employed. That means being able to include more than one different sticky-end (single-stranded overhang) in the system, in order to have at least one difference. In the described figure, three are shown because to create many differences one can either:
   i) Use many different restriction sites (and corresponding restriction enzymes) to encode many different sticky ends (as shown in the Figures)
   ii) Or, use restriction enzymes (REs) that cleave engineered/designed sites, herein illustrated with the Type IIS enzymes, which cleave dsDNA at a site distant from their recognition site (RS). In this case, the cleavage site (CS) can be designed to contain any sequence (since TIIS REs cut at a location a set distance from their RS, rather than cutting at a set sequence) so long as it is at the appropriate location (relative to the RS), which cannot be done with a normal restriction enzyme. By designing the sequence of each CS, a single Type IIS RE can generate many different sticky ends (overhang sequences), depending on the configuration of the RS relative to the designed/engineered/encoded CS.

(2) A labeled probe that can be ligated to the cleaved barcode.
a) For normal restriction enzymes (those that cut at their recognition site), this is straightforward. If a dsDNA molecule is cut with XhoI, it will have an overhang that will ligate with a probe that has the same overhang. The XhoI overhang is well known and consistent for all, and this is the way people have been cloning for decades. The problem is that normal REs will only cleave dsDNA at their respective RS, so only one probe is needed per enzyme being used (in order to distinguish that particular overhang on an OCLS barcode).
b) A defined number of probes is used in each decoding cycle. For example, if there are a possibility of 10 different barcode overhangs in a given cycle, the 10 probe possibilities are added-all 10 will match (that is, all 10 will have overhangs that can bind and ligate to specific exposed overhangs on barcodes), but only at the correct barcode (only if correct base complementarity is generated between the overhang on the barcode and the overhang on the probe). The system is contrived, as with next generation DNA sequencing, and the read out result is what bead(s) or feature(s) have what barcode on them.
c) For the Type IIS ligation probes, the sites for ligation are designed into the barcode sequences, and they correspond to customized (designed) overhangs that are revealed after digestion with the Type IIS restriction enzyme. This is truly scalable multiplexing, because there is no need to find 10 differentially labeled restriction enzymes to make 10 different overhangs for detection by 10 different ligation probes. Here a single enzyme can make all 10 different ligate-able ends that can be recognized by 10 differentially labeled probes.
   i) It is also possible to multiplex numerous (e.g., 10) different probes with only four different fluorophore colors. If only 4 different fluorophores are used, mixing these in combinations to make 10 different distinguishable probes that can be read-out in a single image is necessary. This can be done as follows: when two differentially-labeled probes are mixed in different combinations for each of the individual probe types, it is possible to distinguish 10 different combinations with 4 distinguishable labels (labels 1-4): 1+1, 1+2, 1+3, 1+4, 2+2, 2+3, 2+4, 3+3, 3+4, 4+4. With five different labels, 15 combinations can be distinguished. However, as one cannot distinguish between 1+2 vs 2+1 for example, those will look the same to the observer.
      (1) FIGS. 1A-1B illustrate multiplexing that can go as far as the number of different restriction enzymes (RE) that are used across all cycles in the experiment. The recognition sites (RSs) can be put in different combinations, but this embodiment can be scaled only to a limited level—there is no scalability beyond using a known number of RS/RE pairs, and it is only possible to increase combinations by directly increasing the number of RS/RE pairs used, combinations with further multiplexing of distinguishable visual labels (such as fluorophores).
      (2) FIG. 2, on the other hand, illustrates using the Type IIS barcodes and enzymes to scale up the different possible encoded sticky ends for ligation, by which potentially billions of different beads can be encoded in combinatorial fashion, for example as shown in FIG. 1B, and decoded across cycles of the method, because one enzyme can actually be used to encode many different overhangs (since the “cut” site can be engineered to be a different sequence each time, and since the Type IIS RE recognizes its RS and simply cuts some set distance away, into whatever the sequence is at that distant location). The same enzyme can be used over and over if necessary to encode a billion different combinations. That is also enabled by re-introducing another Type IIS enzyme recognition site into the labeled probe, such that when ligated to the barcode, it creates another Type IIS recognition site within the correct distance from a corresponding encoded “next cleavage site” on the barcode.

(3) An imaging step to determine probe ligation. The imaging step can be carried out using any appropriate means, given the label(s) being used and the sample(s) being analyzed; and

(4) A method and system for decoding the barcodes when attached to beads or other localizable solid surface location(s).
a) The described method is designed to be scalable for encoding when it is performed in cycles. A cycle involves: cleaving the barcode; ligating a labeled probe to the cleaved barcode; and imaging the barcode to which labeled probe has been ligated.
b) When multiple (n) different distinct overhangs are present on a single bead/location, the ligation and imaging step are repeated n times, according to the maximum number of different distinct overhangs present on single beads within the library.

Embodiments of the provided methods for visual molecular barcoding are illustrated in FIGS. 4A-4E and 5A-5E. These methods involve orthogonal cleavage and ligation, and is therefore referred to herein as orthogonal cleavage ligation sequencing (OCLS). The OCLS method uses detectable labels attached to incoming ligation probes, and these labels are used for determining the specific overhang sequences on the beads following cleavage by a restriction enzyme. FIGS. 1A, 1B show one way to accomplish this, which involves the use of multiple different orthogonal restriction enzyme sites (RS1-RS3 shown here). In brief, a series of OCLS tags present on a bead are cleaved through the use of orthogonal restriction enzymes (RE1-RS3 shown here) that specifically recognize one site on each tag. This enables the revealing of specific “sticky ends” (overhangs) for ligation of specific labeled probes (FIG. 4B). Cycles of decoding (ID cycles) involve cleavage, followed by ligation and imaging. Three of these decoding cycles are shown in FIGS. 4A-4E. The cleavage step occurs once per cycle, and the introduction of ligation probes and ligase following cleavage occurs followed by imaging (imaging cycles), will occur according to the number of different enzymes used during cleavage. When performed in various combinations, many beads can be visually encoded and decoded in this way. It is also possible to decode smaller bead libraries using a single ligation and imaging step following cleavage, as shown in FIG. 4E.

FIGS. 5A-5E illustrate an embodiment of OCLS. The stem1 oligos once hybridized, contain a formed dsDNA BsmFI recognition site (RS1); this Type IIS enzyme cleaves 10 bp downstream of its recognition site and leaves a four base, 5′ overhang. The stem2 oligos once hybridized, contain a formed dsDNA BpuEI recognition site (RS2); this Type IIS enzyme cleaves 14 bp downstream of its recognition site and leaves a two base, 3′ overhang (FIGS. 5A and 5B; Unlabeled barcodes orthogonally-cleaved with one or more Type IIS REs: step one). These two enzymes will cut exactly this distance (respectively) downstream of their recognition sites every time they cleave DNA, but the site they cleave is not sequence-specific. This enables coordinated design of orthogonal cleavage sites (overhangs or “sticky ends”) with labeled orthogonal probes that contain compatible overhangs, such that each distinguishably-labeled probe recognizes a specifically-designed cleavage site on the OCLS barcode. dsDNA barcodes (OCLS barcode stems) containing defined 5′- or 3′-overhangs in different order (going from 5′ to 3′ for example) can be encoded by a splitting and pooling ligation strategy to generate libraries of dsDNA barcodes for OCLS. The labeled OCLS ligation probes are covalently-attached to cleaved OCLS barcode oligos by orthogonal ligation, and this newly formed ligation product contains a detectable label that is imaged for identification of the specific overhang sequence. The ligate-able probes also contain a dsDNA region encoding another Type IIS recognition site to enable subsequent cleavage at a different location on the stem oligo following ligation (FIGS. 5C and 5D; Barcodes orthogonally-cleaved with one or more Type IIS REs, identified by orthogonal ligation: step two). This allows for “ratcheting” down the stem oligo toward the bead over cycles, with each cycle containing the steps of: cleavage, ligation and imaging for decoding (FIG. 5E; OCLS preferred: Cleave, (ligate, image)×(number of stems used)−illustrated with two stems: all subsequent cycles). Though illustrated with two stems (and thus, X=2 in FIGS. 5A-5E), it is understood that only one stem may be used, or multiple different stems can be employed in order to increase the multiplexing of the method. In FIGS. 5A-5E, two different stems are shown, so for each cycle the intent would be to see both. Therefore here, this is X2. In the case where more than one stem type is used in the same OCLS decoding workflow, whereby cleavage is performed by more than one enzyme within a single reaction mixture, a cycle of ligation and imaging is implemented for each stem type included. Labeled hybridized dsDNA ligation probes are used for both the BsmFI- and BpuEI-specific stems (stem1 and stem2 respectively), and labeled DNA hairpin ligation probes have also been designed for the BsmFI stem.

In a preferred embodiment, orthogonal ligation probes are comprised of ssDNA hairpin oligos containing detectable labels as shown in FIG. 6. Detectable labels can be attached to oligos through an internal amino modification within the loop region of the hairpin oligo, as shown here. FIG. 6 illustrates one cycle of decoding using hairpin probes for OCLS. In the graph in FIG. 6, data obtained from imaging beads following a first cleavage event, following ligation of a correct OCLS hairpin probe labeled with Alexa Fluor 488 to the cleaved OCLS stem barcode, and following a second cleavage event were graphed beside one another. Here, and as disclosed throughout this application, the incoming OCLS probe not only distinguishes the correct overhanging ligation site, but also encodes another restriction site. In this example, the enzyme recognition site (RS) is recognized by a Type IIS restriction enzyme (RE). Therefore, as shown here, a second digestion removes detectable label from the OCLS stem barcode, and reveals a subsequent encoded sticky end. This prepares the OCLS stem oligo for a subsequent round of decoding by orthogonal ligation. The use of hairpin oligos is beneficial in many ways. Primarily, these benefits include 1) the hybridized strands of a ssDNA hairpin oligo are less prone to de-hybridization than a pair of hybridized, antiparallel oligos paired as a dsDNA duplex species, and 2) the hairpin structure ensures all ligation products do not contain an open 5′-end. This will be important when decoding large libraries of beads using the OCLS method as described here, because incomplete digestion by the restriction enzyme, as well as incomplete ligation by the ligase enzyme, will result in errors during subsequent cycles. To remove un-ligated stem species following the ligation step, a 5′-exonuclease such as lambda exonuclease, which recognizes phosphorylated 5′-ends of oligo nucleotides for removal of DNA by digestion. In between the final imaging step of each cycle, lambda exonuclease can be introduced, which will “clean-up” any remaining un-ligated stems (those which did not receive a hairpin probe). In this case the ligated species will not contain an open 5′-end, and will be protected from the 5′-exonuclease activity of lambda exonuclease. This operational “clean-up” step can also be accomplished by using labeled dsDNA probes containing labels at their 5′-ends (blocking any 5′-exonuclease activity) or that do not contain the 5′-phosphate group, or are otherwise blocked at their 5′-ends.

In other embodiments, one can use the same enzyme for decoding of all stems by OCLS in a reaction milieux. For example, encoded ligation sites on stems or on probes can be revealed by digestion with RNAse H as the cleavage agent, which will enable encoding of sticky ends (overhanging bases) by design. In this case, incoming labeled probes for orthogonal ligation will contain complementary sticky ends for the purpose of decoding the revealed bases on stems following digestion with RNAse H. This approach can be used in substitution of a single cycle of OCLS, as described here for example.

As used herein, “orthogonal” refers to a component in a multicomponent system that has chemical reactivity with a particular reagent under a specific set of reaction conditions while at least one other component in the multicomponent system has limited or no reactivity with the reagent, even though all components in the multicomponent system are present in the same milieu.

As used herein, “orthogonal reactivity” refers to a component in a multicomponent system that has chemical reactivity with a particular reagent under a specific set of reaction conditions while at least one or more components in the system does not, even though all the components in the system are present in the same milieu. Likewise, “orthogonally reactive” refers to a material having orthogonal reactivity.

The phrase “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Detectable labels include a bioluminescent label, a biotin/avidin label, a chemiluminescent label, a chromophore, a coenzyme, a dye, an electro-active group, an electro-chemiluminescent label, an enzymatic label, a fluorescent label, a latex particle, a magnetic particle, a quantum dot, a Raman label, a metal, a metal chelate, a phosphorescent dye, a protein label, a radioactive isotope, element or moiety, and a stable radical. Visually detectable labels are those labels the detection of which involves detection of wavelength(s)/photons of electromagnetic energy (such as light), for instance through imaging and the like. In certain embodiments, fluorescent labels are preferred.

“Fluorescence” refers to the emission of visible light by a substance that has absorbed light of a different wavelength. In some embodiments, fluorescence provides a non-destructive means of tracking and/or analyzing biological molecules based on the fluorescent emission at a specific wavelength. Proteins (including antibodies), peptides, nucleic acid, oligonucleotides (including single stranded and double stranded oligonucleotides), and so forth may be “labeled” with any of a variety of extrinsic fluorescent molecules referred to as fluorophores. Isothiocyanate derivatives of fluorescein, such as carboxyfluorescein, are an example of fluorophores that may be conjugated to proteins (such as antibodies for immunohistochemistry) or nucleic acids. In some embodiments, fluorescein may be conjugated to nucleoside triphosphates and incorporated into nucleic acid probes (such as “fluorescent-conjugated primers”) for in situ hybridization.

Direct and indirect attachments (e.g., of a detectable label to an oligonucleotide or other substance) can include covalent bonds or non-covalent interactions. Covalent bonds include the sharing of electrons in a chemical bond. Non-covalent interactions include dispersed electromagnetic interactions such as hydrogen bonds (such as occurs between paired strands of nucleic acids), ionic bonds, van der Waals interactions, and hydrophobic bonds.

The term “linker” refers to one or more of a nucleotide, a nucleotide analog, an amino acid, a peptide, a polypeptide, a polymer, or a non-nucleotide chemical moiety that is used to join two molecules to each other. A linker may be used to join a nucleic acid (such as a barcode) with a support, a detection agent with a nucleic acid (such as a probe), and so forth. In certain embodiments, a linker joins two molecules via enzymatic reaction or chemistry reaction (e.g., click chemistry).

As used herein, “next generation sequencing” (NGS) refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (see e.g., Service, Science 311:1544-1546, 2006).

As used herein, “single molecule sequencing” or “third generation sequencing” refers to next-generation sequencing methods wherein reads from single molecule sequencing instruments are generated by sequencing of a single molecule, generally a molecule of DNA. Unlike next generation sequencing methods that rely on amplification to clone many DNA molecules in parallel for sequencing in a phased approach, single molecule sequencing interrogates single molecules (e.g., of DNA) and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

By way of example, an NGS capture barcode is an oligonucleotide that contains a capture region, which is a stretch of nucleotides that is complementary to oligonucleotides used a biological, chemical, or biochemical assay or that are found within a biological sample. Generally, capture oligoes include (going from 3′ to 5′) a 3′ capture region, and a UMI, and then here they have a region that corresponds to the visual barcode information (such that they are operationally coupled on the same bead), then they have a 5′ PCR handle. Other elements within a capture oligonucleotide are described in co-owned International Application No. PCT/US24/17772 entitled “COMPOSITIONS AND METHODS FOR MOLECULAR BARCODING”.

As used herein, the terms “solid support”, “solid surface”, or “solid substrate”, or “sequencing substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof.

For example, when the solid surface is a bead, the bead can include a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from 0.2 micron to 200 microns, or from 0.5 micron to 5 micron. In some embodiments, beads can be 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from 1 nm to 500 nm in diameter, for example, between 1 nm and 20 nm, between 1 nm and 50 nm, between 1 nm and 100 nm, between 10 nm and 50 nm, between 10 nm and 100 nm, between 10 nm and 200 nm, between 50 nm and 100 nm, between 50 nm and 150, between 50 nm and 200 nm, between 100 nm and 200 nm, or between 200 nm and 500 nm in diameter. In some embodiments, the nanoparticles can be 10 nm, 50 nm, 100 nm, 150 nm, 200 nm, 300 nm, or 500 nm in diameter. In some embodiments, the nanoparticles are less than 200 nm in diameter.

The term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

A captured probe, as the term is used herein, is an orthogonal ligation probe that is covalently linked to the OCLS barcode by a ligation event. Though exemplified herein using enzymatic ligation, the use of chemical ligation to capture probes on OCLS barcodes is also contemplated. For example, there are a variety of chemical reactions that are known in the art that can be used for chemical ligation of DNA. Each of the two molecules being joined together by chemical ligation must bear one of the reaction components for the chemical ligation, and the reaction must only occur at very high proximity (such that the reaction only proceeds when the overhang of the orthogonal ligation probe binds by specific base pair-mediated hydrogen bonding to the overhang on the stick end of an OCLS barcode, when the sequence of the overhang of the orthogonal ligation probe is the reverse complement of the sequence of the overhang of the OCLS barcode). This can be dialed-in by adjusting reaction conditions, such that the chemical ligation occurs only when two different DNA molecules (like a probe and a barcode described here), come in very close proximity to one another, which occurs when there is correct base complementarity between the two compatible ends (matching probe and barcode overhangs here).

In embodiments, the binding of a partially single-stranded probe with a partially single stranded OCLS barcode, where at least a portion of the single-stranded elements are sticky end overhangs generated by restriction enzyme digestion, occurs when the two overhangs have matching base complementarity. In order for the two nucleic acid molecules to be ligated to each other, the cognate single stranded sequences must be the reverse complement of each other. Correct base complementarity here literally means that one is the reverse complement of the other. When an overhang on a probe has come in contact with a correctly matching overhang on a cleaved barcode, the two are and must be the reverse complement of each other for the ligation to occur-thereby “cementing” the association between the “correct” probe and the OCLS barcode. It is because the initial interaction will likely be a very “touch down” transient type of interaction between the correct matching sequences; chemical ligation of action of a ligase enzyme is employed to seal the probe to its cognate barcode.

Embodiments provide methods of orthogonal cleavage-ligation sequencing (OCLS) of visual barcodes. This method is disclosed herein, including in the accompanying Figures. Although identimer chains (such as those employed in OCS, as described in WO2022/187719) may be composed of polymers other than nucleic acids (such as peptide linkers using orthogonal proteases or chemical linkers using orthogonal chemical cleaving agents), the barcodes used for OCLS as described are composed of dsDNA. Visually decoded barcodes used in OCLS workflows are also built over rounds of splitting and pooling, but the segments making up OCLS barcodes are not labeled (as they are with identimers). Each segment of the OCLS library is ligated to a previous segment to build a chain, and these segments can be co-encoded along with a NGS capture barcode as outlined above. In similar fashion to splitting and pooling procedures outlined in WO 2022/187719, 100 or more different wells can be used in each round to build the bead libraries, over several rounds of splitting and pooling.

For decoding, the OCLS method involves the use of at least one, but optionally two or more orthogonal restriction enzymes (REs), such as Type IIS enzymes (for example, those used in FIGS. 4A-4E and FIGS. 5A-5E, and the corresponding legends provided herein), in a single reaction mixture. This step is followed by specific orthogonal ligation of differentially-labeled probes, which enables visual distinguishing of the barcodes at each feature. This process is repeated over cycles as outlined in the figures and legends.

By way of comparison to orthogonal cleavage sequencing (OCS; as described in WO 2022/187719), embodiments of OCLS as described herein: use visual barcodes that do not contain any visual labels prior to the introduction of cleavage agents; involve the introduction of more than one cleaving agent at a time; involve introducing detectable labels during each cycle, and are determined by a gain in signal; and include both orthogonal cleavage and orthogonal ligation steps.

A major benefit provided in embodiments of the methods herein is use of Type IIS enzymes (which recognize asymmetric DNA sequences and cleave outside of their recognition sequence). If regular restriction enzymes are used instead, they will cleave at their recognition site, which always leaves the same overhang (that is, the same RE will always leave the same overhang). That means to multiplex with regular REs, one must use a different RE for each “orthogonal” sticky-end for ligation. For instance, 10 different REs would be needed at each cycle to decode 10 different sequences by orthogonal ligation. By instead using Type IIS enzymes, the enzyme recognition site is a known number of bases away from the cleavage site of the RE, which allows design of the oligonucleotides such that a single (Type IIS) enzyme can create many different “orthogonal” cleavage/ligation sites. The label(s) associated with each labeled ligation step are pre-determined (which labels correspond to which ligation sites are known in advance), which allows for one to determine the code or “series of designed ligation sites” as these are coordinated with the colors following ligation and imaging. Type IIS REs are known in the art, including the more than 50 described online at neb.com/tools-and-resources/selection-charts/type-iis-restriction-enzymes. This online resource provides the recognition sequence and other characteristics for each of the listed Type IIS REs, which include: AcuI, AlwI, BaeI, BbsI, BbsI-HF, BbvI, BccI, BceAI, BcgI, BciVI, BcoDI, BfuAI, BmrI, BpmI, BpuEI, BsaI-HF®v2, BsaXI, BseRI, BsgI, BsmAI, BsmBI-v2, BsmFI, BsmI, BspCNI, BspMI, BspQI, BsrDI, BsrI, BtgZI, BtsCI, BtsI-v2, BtsIMutI, CspCI, EarI, EciI, Esp3I, FauI, FokI, HgaI, HphI, HpyAV, MbolI, MlyI, MmeI, MnlI, NmeAllI, PaqCI, PleI, SapI, and SfaNI.

Visually decoded barcodes used in OCLS workflows, similar to OCS barcodes, are also built over rounds of splitting and pooling (see the description in WO 2022/187719). However, the segments making up OCLS barcodes are not labeled (as they are with OCS identimers). Each segment of the OCLS library is bound to a bead or is ligated to a previous segment to build a chain, and these segments optionally can be co-encoded along with a NGS capture barcode as described for OCS (WO 2022/187719).

For decoding, embodiments of the OCLS method use two or more orthogonal REs, such as Type IIS enzymes (for example, those described herein), in a single reaction mixture (for instance, simultaneously). This step is followed by specific orthogonal ligation of differentially-labeled probes, which enables visual distinguishing of the barcodes at each feature. This process is repeated over cycles as described in the figures and legends.

In instances that employ only one enzyme for decoding OCLS barcodes at each cycle, each cycle would simply involve: cleave, ligate, image. However, when two different stems are used (as illustrated in FIGS. 5A-5E), which are recognized by two different Type IIS enzymes, each cycle involves: cleave with the two enzymes, ligate to the first stem type (as illustrated, all labeled ligation probes that recognize the first overhang type are introduced), an imaging step is performed to determine the labels associated with the first overhang type, followed by a second ligation step (all labeled ligation probes that recognize the second overhang type are introduced) which enables visual determination of the second overhang type during a second imaging step. Because the ligation probes (now products) contain another Type IIS enzyme site, the next cleavage cycle removes the previously ligated labels, prior to addition of new labels upon the next ligation cycle.

In this way, one ratchets down the barcode by alternating between cleavage, ligation, and imaging. The ligation and imaging steps are what are repeated (within each cycle) corresponding to the number of different stem types being used. That is because ligation probes introduced after each cleavage event (when using more than one enzyme during the cleavage step), are connected to an image step to enable deconvolution. So that gives rise to the approach of: cleave, ligate, image, ligate, image in sequence of downstream steps.

In operation, the system operates to [ligate and image]×the number of different stems used, and that is because the ligation probes contain redundant labels across the two (or however many) stem types. An image is obtained between each ligation event to determine the overhang sequence. The labels on the ligation probes reveal what the overhang sequence was.

### Methods of Use

The visual barcoding technologies provided herein have myriad and diverse uses, including for visual identification, for instance of beads or molecules tagged with the described barcodes. For example, different antibodies tagged with this approach can be used in visual cyclicIF-like workflows. Likewise, these molecular barcodes can be used on beads bearing drug or other libraries in high throughput biochemical and cell-based screens for the rapid visual identification of drugs, enabling their correlation with visual reporter activation events and/or omics-based read-outs. Other implementations include their use in generating a spatially-encoded array when attached to beads and used for encoding NGS capture oligos (refer to the analogous system described in co-owned International Application No. PCT/US24/17772 entitled “COMPOSITIONS AND METHODS FOR MOLECULAR BARCODING”, which is incorporated by reference herein for all it teaches). These are not intended to be limiting example uses, and one of ordinary skill will appreciate that the provided OCLS barcodes, beads or other solid supports loaded with such barcodes, and labeled orthogonal ligation probes for use with such barcodes, are amenable to many other uses and applications.

### Kits and Articles of Manufacture

Also provided herein are kits and articles of manufacture that include components for OCLS analysis using a method and/or components described herein. In some embodiments, the kits further contain other reagents for treating and analyzing biological samples and/or molecules, such as nucleic acids, polynucleotides, proteins, polypeptides, or peptides. The kits and articles of manufacture may include any one or more of the reagents and components used in a provided method. In some embodiments, the kit includes one or more of beads or other support surface(s), OCLS barcodes(s) (optionally, already attached to beads or other support surface(s)), individual orthogonal ligation probes or collections (sets) of such probes (which optionally are designed to pair with, and may be packaged with, cognate OCLS barcode(s), processing or reaction compounds or solutions for use in a OCLS barcode encoding or decoding method.

In some embodiments, the kit also includes one or more buffers or reaction fluids useful for or necessary for any of the reactions to occur. Buffers such as wash buffers, reaction buffers, binding buffers, elution buffers and the like are known to those or ordinary skill in the arts. In some embodiments, the kits further include buffer(s), and one or more additional components to accompany other reagents described herein. The reagents, buffers, and other components may be provided in vials (such as sealed vials), vessels, ampules, bottles, jars, flexible packaging (e.g., sealed Mylar or plastic bags), and the like. Any of the components of the kits may be sterilized and/or sealed.

In addition to above-mentioned components, the subject kits may further include instructions for using the components of the kit to practice the subject methods, such as instructions for sample preparation, visual barcode encoding, visual barcode decoding, signal detection and obtention, and/or analysis of data obtained from the method(s). The kits described herein may also include other materials, such as those that may be deemed desirable from a commercial and user standpoint, including other buffers, diluents, filters, syringes, and/or package inserts with instructions for performing at least one of the methods described herein.

Any of the above-mentioned kit components, and any molecule, molecular complex or conjugate, reagent (e.g., chemical or biological reagents), agent, structure (e.g., support, surface, particle, or bead), reaction intermediate, reaction product, binding complex, or any other article of manufacture disclosed and/or used in the exemplary kits and methods, may be provided separately or in any suitable combination in order to form a kit.

Also contemplated are devices useful to apply chemicals or compositions or washes/solutions to immobilized visual barcodes, including during decoding reactions, and analyzing and gathering data from such processes through use of a provided methods (such as washes, buffers, reaction solutions, and the like), including flow throw fluid devices and micro-fluidic devices.

Devices for detecting and measuring spectral characteristics of the labeled, captured orthogonal ligation probes, such as devices for detecting visually-detectable labels, are also contemplated.

Further embodiments are analysis software and amino acid deconvolution databases prepared using, or intended to be used with, analysis/detection/quantification/sequencing methods that employ OCLS barcodes and/or orthogonal ligation probes as provided herein.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A visual barcode including: at least two or more double-stranded DNA (dsDNA) oligonucleotide segments (cassettes) functionally linked linearly to each other, and each including within the sequence of the dsDNA segment: a recognition site (RS) for a specific restriction endonuclease (RE), a designed cleavage site (CS), or both a RS and a CS; and an untethered end at one end of the visual barcode.

2. The visual barcode of embodiment 1 or any other barcode embodiment, tethered to a solid substrate by a flexible linker attached at or near an end of the dsDNA of the visual barcode that is not the untethered end.

3. The visual barcode of embodiment 1 or any other barcode embodiment, which does not include a visually detectable label.

4. The visual barcode of embodiment 1 or any other barcode embodiment, wherein the RE is a Type IIS restriction endonuclease, and the CS does not overlap the corresponding RS.

5. The visual barcode of embodiment 1 or any other barcode embodiment, wherein at least one of the dsDNA segments includes a designed CS and the visual barcode includes a RS specific for a Type IIS RE, positioned appropriately such that the cognate Type IIS RE can cut the designed CS based on its position relative to the RS.

6. The visual barcode of any one of embodiments 1-5, as illustrated in FIG. 1A or FIG. 1B or essentially as described herein.

7. A collection of visual barcodes of any one of embodiments 1-6 or any other barcode embodiment, wherein the collection includes a plurality of visual barcodes each of which includes a different set of dsDNA segments having different recognition sites (RSs) for specific restriction endonucleases (REs), designed cleavage sites (CSs), or both.

8. The collection of visual barcodes of embodiment 7, wherein at least two of the different visual barcodes are tethered to the same solid substrate.

9. An orthogonal cleavage-ligation sequencing (OCLS) barcode including two or more dsDNA segments (cassettes), each dsDNA segment containing a recognition site (RS) for a specific restriction endonuclease (RE), and one or more overlapping region(s) configured to permit ligation to flanking dsDNA segments to form a chain of segments, which chain of segments constitutes the OCLS barcode.

10. A visually detectable orthogonal ligation probe including: a fully or partially double-stranded DNA oligonucleotide, having a 3′ or 5′ overhang of at least two nucleotides, the sequence of which includes: a recognition site (RS) for a specific restriction endonuclease (RE), a designed cleavage site (CS), or both a RS and a CS; and covalently attached to the fully or partially dsDNA oligonucleotide, a visually detectable label.

11. The visually detectable orthogonal ligation probe of embodiment 10 or any other probe embodiment, wherein the fully or partially double-stranded DNA oligonucleotide includes: a linear double-stranded DNA oligonucleotide having a 5′ overhang; a linear double-stranded DNA oligonucleotide having a 3′ overhang; a hairpin stem-loop configured single-stranded DNA oligonucleotide having a 5′ overhang; or a hairpin stem-loop configured single-stranded DNA oligonucleotide having a 3′ overhang.

12. The visually detectable orthogonal ligation probe of embodiment 10 or any other probe embodiment, wherein the visually detectable label is attached to the oligonucleotide by way of a flexible linker.

13. The visually detectable orthogonal ligation probe of embodiment 10 or any other probe embodiment, wherein the visually detectable label includes one or more of a fluorescent label, a bioluminescent label, a chemiluminescent label, a chromophore, a quantum dot, a Raman label, a biotin moiety, or a radioactive isotope.

14. The visually detectable orthogonal ligation probe of embodiment 10 or any other probe embodiment, wherein the RE is a Type IIS restriction endonuclease, and RS is specific for that Type IIS RE.

15. The visually detectable orthogonal ligation probe of embodiment 10 or any other probe embodiment, including a RS specific for a Type IIS RE, positioned appropriately so the cognate Type IIS RE can cut a designed CS based on its position relative to the RS.

16. The visually detectable orthogonal ligation probe of any one of embodiments 10-15 or any other probe embodiment, as illustrated in FIG. 3A or FIG. 3B or essentially as described herein.

17. A collection of visually detectable orthogonal ligation probes of any one of embodiments 10-16 or any other probe embodiment, wherein the collection includes a plurality of visually detectable orthogonal ligation probes each of which includes a different recognition site (RSs) for specific restriction endonucleases (REs), or both a different RS and a different cleavage site (CS).

18. The collection of visually detectable orthogonal ligation probes of embodiment 17 or any other collection embodiment, wherein at least two of the different detectable orthogonal ligation probes include visually distinguishable detectable labels.

19. The collection of visually detectable orthogonal ligation probes of embodiment 18 or any other collection embodiment, including a plurality of different probes each of which is configured such that cleavage of that probe with the RE produces an overhang having a sequence different from at least 5, at least 7, at least 10, at least 12, at least 15, or more than 15 other probes in the collection.

20. An orthogonal cleavage-ligation sequencing (OCLS) oligonucleotide pair, including: a visual barcode, including: at least two or more double-stranded DNA (dsDNA) oligonucleotide segments (cassettes) functionally linked linearly to each other, and each including within the sequence of the dsDNA segment: a recognition site (RS) for a specific restriction endonuclease (RE), a designed cleavage site (CS), or both a RS and a CS; and an untethered end at one end of the visual barcode; and a visually detectable orthogonal ligation probe, including: a fully or partially double-stranded DNA oligonucleotide, having a 3′ or 5′ overhang of at least two nucleotides, the sequence of which includes: a recognition site (RS) for a specific restriction endonuclease (RE), or both a RS and a cleavage site (CS); and covalently attached to the fully or partially dsDNA oligonucleotide, a visually detectable label; wherein cleavage of the RS or the CS in the visual barcode produces a single-stranded “sticky end” overhang having a sequence with full complementary to a sticky end (overhang) produced by cleavage of the RS in the visually detectable orthogonal ligation probe.

21. A set of OCLS oligonucleotides pair of embodiment 20, wherein each pair of visual barcode and visually detectable orthogonal ligation probe have a different fully complementary sequence overlap, and each visually detectable orthogonal ligation probe includes a different visually distinguishable detectable label.

22. A method of encoding a visual barcode, including: contacting a double-stranded DNA (dsDNA) oligonucleotide tethered at a first end to a solid support, which dsDNA oligonucleotide has a single-stranded overhang at a second, untethered end, with a first dsDNA segment having a first overhanging end compatible for binding to the single-stranded overhang of the tethered dsDNA oligonucleotide and a second overhanging end, and including within the sequence of the first dsDNA segment: a recognition site (RS) for a specific restriction endonuclease (RE), a designed cleavage site (CS), or both a RS and a CS; which contacting occurs under conditions sufficient to allow specific hybridization of the single-stranded overhang of the first dsDNA segment to the single-stranded overhang of the tethered dsDNA oligonucleotide; ligating the first dsDNA segment to the tethered dsDNA oligonucleotide, to form a first captured barcode segment, which includes the second overhanging end of the first dsDNA segment; contacting the first captured barcode segment with a second dsDNA segment having a first overhanging end compatible for binding to the single-stranded overhang of the first captured barcode segment oligonucleotide and a second overhanging end, and including within the sequence of the second dsDNA segment: a RS for a specific RE different from the RS/RE in the first dsDNA segment, a designed CS different from the designed CS in the first dsDNA segment, or both; and ligating the second dsDNA segment to the first captured barcode segment, to form a second captured barcode segment, which includes the second overhanging end of the second dsDNA segment, which first captured barcode segment and second captured barcode segment constitute the visual barcode.

23. The method of embodiment 22 or any other method embodiment, further including repeating the contacting and ligating steps one or more additional times, each time attaching an additional dsDNA segment to the captured barcode segments, to form the visual barcode.

24. The method of embodiment 22 or embodiment 23 or any other method embodiment, wherein at least one of the dsDNA segments includes a designed CS and the visual barcode includes a RS specific for a Type IIS RE, positioned appropriately so the cognate RE can cut the designed CS based on its position relative to the RS.

25. The method of any one of embodiments 22-24 or any other method embodiment, as illustrated in FIG. 1A or FIG. 1B or as described herein.

26. The method of embodiment 24 or any other method embodiment, wherein the visual barcodes are built using rounds of splitting and pooling using unlabeled DNA segments.

27. The method of embodiment 26 or any other method embodiment, wherein one or more rounds of splitting and pooling include(s): ligating one barcode cassette at a time onto a bead; splitting the resultant beads into individual compartments, optionally wells of a plate; ligating a different first compartment-specific barcode cassette onto the beads in each individual compartment, to yield a collection of beads containing different pairs of two barcode cassettes; washing the beads containing two barcode cassettes; pooling the beads containing two barcode cassettes; splitting the pooled beads containing two barcode cassettes into individual compartments, optionally wells of a plate; and repeating the ligating, washings, pooling, and splitting steps to increase diversity of the set of barcodes.

28. The method of any one of embodiments 22-27 or any other method embodiment, wherein the visual barcode includes a contiguous chain of dsDNA segments (cassettes), or the visual barcode includes at least two separate cassettes attached directly and separately to the solid support.

29. The method of any one of embodiments 22-28 or any other method embodiment, wherein the segments are co-encoded along with a next-generation-sequence (NGS) capture barcode.

30. A visual barcode made by the method of any one of embodiments 22-29.

31. A method of decoding a visual barcode, including: contacting, in a milieu, at least one double-stranded DNA (dsDNA) orthogonal cleavage-ligation sequencing (OCLS) barcode including at least one restriction site (RS), with a restriction endonuclease (RE) that recognizes that RS, under conditions sufficient for the RE to cleave the dsDNA OCLS barcode, which RS/RE cleavage results in a single-stranded overhang to produce a partially single-stranded (ss) DNA-partially dsDNA OCLS barcode; contacting the partially ssDNA-partially dsDNA OCLS barcode with at least one orthogonal ligation probe including a dsDNA oligonucleotide including an overhang at a first end and a visually detectable label, under conditions sufficient for the overhang of the orthogonal ligation probe to bind by base pair-mediated hydrogen bonding to the overhang on the partially ssDNA-partially dsDNA OCLS barcode if the sequence of the overhang of the orthogonal ligation probe is the reverse complement of the sequence of the overhang of the partially ssDNA-partially dsDNA OCLS barcode; if base pair-mediated binding occurs, ligating the orthogonal ligation probe to the partially single-stranded (ss) DNA-partially dsDNA OCLS barcode to produce a captured probe; and detecting presence, absence, and/or quantity of captured probe by imaging the visually detectable label.

32. The method of embodiment 31 or any other method embodiment, further including repeating the contacting/cleavage, contacting/base pair-mediated binding, ligating, and detecting steps cycle one or more times, where each additional cycle involves a cleavage of the dsDNA OCLS barcode at a different cleavage site (CS), base pair-mediated binding of a different orthogonal ligation probe, and/or detection of the presence, absence, and/or quantity of a different labeled captured probe.

33. The method of embodiment 31 or any other method embodiment, wherein one or more of: the dsDNA OCLS barcode is attached to a bead or other solid surface; a plurality of different dsDNA OCLS barcodes are attached to a single bead or single address on another solid surface; the dsDNA OCLS barcode includes more than one non-overlapping RSs; at least one RS in the dsDNA OCLS barcode is recognized by a Type IIS RE, and cleavage occurs at a predetermined location outside of the RS; at least one RS in the dsDNA OCLS barcode is recognized by a RE that cleaves within the RS; or the visually detectable label includes at least one of a fluorescent label, a bioluminescent label, a chemiluminescent label, a chromophore, a quantum dot, a Raman label, or a radioactive isotope.

34. The method of embodiment 32, or any other method embodiment wherein one or more of: the orthogonal ligation probe includes a single stranded DNA oligonucleotide having a stem-loop hairpin structure, wherein the overhang is at the end of the stem of the hairpin.

35. The method of embodiment 34 or any other method embodiment, after ligating, further including contacting the milieu including the captured probe with a 5′-exonuclease.

36. The method of embodiment 32 or any other method embodiment, including one or more of: contacting the at least one dsDNA OCLS barcode sequentially with two or more REs that each recognize a different, non-overlapping RS within the dsDNA OCLS under conditions sufficient for each RE to cleave the dsDNA OCLS barcode, each of which RS/RE cleavage results in a single-stranded overhang to produce a partially single-stranded (ss) DNA-partially dsDNA OCLS barcode; the ligating includes chemical ligation; or the ligating includes enzyme-mediated ligation.

37. The method of embodiment 31 or any other method embodiment, including: contacting two or more dsDNA OCLS barcodes, each including at least one RS, with a RE that recognizes that RS under conditions sufficient for the RE to cleave the dsDNA OCLS barcode, which RS/RE cleavage results in a single-stranded overhang to produce a partially single-stranded (ss) DNA-partially dsDNA OCLS barcode, wherein the RS/RE is different for each dsDNA OCLS barcode.

38. The method of embodiment 37 or any other method embodiment, including contacting the two or more dsDNA OCLS barcodes with two or more orthogonal REs.

39. The method of embodiment 38, wherein the contacting with two or more orthogonal REs is simultaneous or sequential.

40. The method of embodiment 39 or any other method embodiment, wherein following contacting with the one or more orthogonal REs, differentially-labeled probes are ligated using specific orthogonal reactions.

41. The method of embodiment 40 or any other method embodiment, wherein the specific orthogonal ligation of differentially-labeled probes enables visual distinguishing of the barcodes at each feature in an array of visual barcodes.

42. The method of embodiment 31 or any other method embodiment, wherein ligating the orthogonal ligation probe to the partially ssDNA-partially dsDNA OCLS barcode adds a new RS to the resultant a captured probe.

43. A method of decoding visual barcodes using orthogonal cleavage-ligation sequencing (OCLS) essentially as described herein.

44. An improved system for molecular barcoding, including repeated cycles of labeling, orthogonal cleavage, ligation, and imaging in order to identify individual features, wherein the orthogonal cleavage includes cleaving a double-stranded DNA barcode with a Type IIS Restriction Endonuclease.

45. The system of embodiment 44, wherein two or more labeling and orthogonal probe ligation identification cycles occur in series, or occur concurrently.

46. Orthogonal cleavage-ligation sequencing (OCLS) system, as illustrated in FIGS. 5A-5E.

### Example 1: Orthogonal Cleavage-Ligation Sequencing (OCLS) Using a Labeled, ssDNA Hairpin Probe

This Example describes an exemplary system and method of OCLS, using as probe a labeled, single-stranded DNA that forms a hairpin structure.

To demonstrate a single cycle of OCLS (cleavage, ligation, cleavage), the oligo pair TIIS stem1 (BsmFI) and THIS stem1 comp were first annealed in 1×NHS Conjugation Buffer (NCB: 100 mM NaPO₄, pH 8.5). Annealing was performed by mixing these oligos at an equimolar ratio (at a final concentration of 200 μM), subjecting them to 95° C. in a heat block for 5 minutes, then allowing the block to cool to RT over a period of at least 1 hour. The annealed dsDNA was then biotinylated (via the primary amino group provided on the 5′-end of the TIIS stem oligo) by adding NHS-LC-Biotin (APExBIO A8004) at a final concentration of 2 mM, to the solution containing 200 μM duplexed oligo. The biotinylation reaction was allowed to proceed overnight at room temperature, then quenched by adding 1M tris buffer (pH 7.5) to a final concentration of 50 mM. The quenched reaction was desalted and buffer-exchanged into 10 mM Tris pH 7.5 buffer containing 80 mM NaCl by using two successive Zeba™ Spin Desalting Columns, 7K MWCO (Thermo Fisher 89882). The biotinylated and annealed oligo was then bound to MyOne T1 streptavidin 42ynabeads (ThermoFisher 65601) at a concentration of 200 nM annealed oligo mixed with 0.1 mg/ml MyOne T1 beads, in 1× streptavidin bind buffer (SBB: 10 mM Tris pH 7.5; 500 mM NaCl). The DNA hairpin ligation probes (TIIS s1hp1 and s1hp3) were labeled with Alexa Fluor 488 NHS Ester (Thermo Fisher Scientific A20000) and Alexa Fluor 555 NHS Ester (Thermo Fisher Scientific A20009) in the same way as described above, but without the need for prior annealing.

MyOne T1 beads coated with the TIIS stem1 oligo (0.25 mg/ml beads in a 200 μl volume) were incubated with BsmFI (NEB R0572L) which had been desalted on a Zeba™ Spin Desalting Columns, 7K MWCO (Thermo Fisher 89882) and then eluted in 1× rCutSmart™ buffer to remove glycerol prior to usage. The BsmFI enzyme (40 μl) was added to the 200 μl of MyOne T1 beads at 0.25 mg/ml) and incubated at 37° C. for 40 minutes (round one digestion). At the end of 40-minutes, the sample was subjected to six washes (2 washes in 1× rCutSmart™ buffer, 2 washes in 10 mM Tris-HCL pH 7.5/500 mM NaCl, and 2× in 1×T4 DNA ligase buffer (NEB M0202S)). During one of the 200 μl Tris-NaCl washes, a 30 μl sample of suspended beads was collected and set aside (Sample 1). Following these washes the BsmFI digested Stem1 oligo-coated beads were subject to a ligation reaction containing 1 μM TIIS s1hp1 labeled with Alexa-488. In this ligation reaction, T4 DNA ligase (NEB M0202S) was added at 10 units/μl and T4 PNK (NEB M0201S) was added at 0.5 units/μl. This ligation was allowed to incubate at RT for 40 minutes. At this point, the reaction was subject to the same 6× washes as mentioned above but during one of the 170 μl Tris-NaCl washes a 30 μl sample of suspended beads was collected and set aside (Sample 2). Following these washes the beads were then subjected to a second round of digestion with BsmFI, with the beads and enzyme being kept at the same concentration as they were in the first digestion described above. After two washes in 1×rCutSmart™ buffer, two additional washes were performed using 140 μl of 10 mM Tris-HCL pH 7.5/500 mM NaCl. On the second of these final washes, a 30 μl sample of suspended beads was collected (Sample 3).

The three bead samples were analyzed via two separate methods. In the first, 2.5 μl of each of the bead samples were bound to a biotin coated glass slide and then mounted for fluorescence microscopy on a Leica THUNDER widefield microscope equipped with a 20×0.8 NA PLAN-apo objective. Using the exact same imaging parameters, images of the three bead samples were acquired using a 488 LED light source and the images were quantified for A488 bead fluorescence intensity using the Volocity® image analysis package from Qurom Technologies Inc. This analysis demonstrated that following the ligation reaction with THIS s1hp1 labelled with Alexa-488, the background subtracted sum intensity of the beads increased 42-fold (p<0.0001) (FIG. 6 graph) over the BsmFI digested stem1 oligo-bearing beads, demonstrating that the stem1 oligo was digested on the beads with BsmFI, and that the Alexa-488 labeled s1hp1 probe containing the cognate overhang was able to ligate to this oligo. Because the Alexa-488 labeled s1hp1 probe introduced a new BsmFI site onto the stem1 oligo following ligation, which was appropriately distanced from an encoded cleavage site downstream of the A488 label, a second productive cleavage event should remove the label from the stem1 oligo. Following a second digestion with the BsmFI enzyme, beads were found to have returned to baseline fluorescence levels, demonstrating that BsmFI had recognized the BsmFI site introduced upon ligation of the Alexa-488 labeled s1hp1 probe (p<0.0001) and had cleaved the stem1 oligo at the encoded “next correct” cleavage site. These results show that over a cycle, the enzyme has “ratcheted” down the stem1 oligo to the next designed sequence for ligation of a “next correct” labeled probe. These results demonstrate one round of OCLS performed on an OCLS barcode oligo that is bound to a bead.

The second method employed to analyze these results was performed on a 10% TBE-urea gel (Thermo Fisher). Briefly, 15 μl of each of the above bead samples (Samples 1-3) was mixed with 15 μl of 2×formamide loading dye (95% formamide, 10 mM EDTA) and heated to 95° C. for 5-minutes and then placed directly on wet ice. In addition, a fourth sample containing beads coated with undigested stem1 oligo was also generated and prepared for loading onto the denaturing gel as described above. These four samples were run on the gel for 1.5 hours at 150 volts in 1×TBE buffer, and then imaged on a ThermoFisher iBright system that allows for documentation of SYBR gold stained gels, and has illumination modules for imaging 488, 550, 647 and 750 dyes. Analysis of this gel demonstrated that following digestion of beads coated with the stem1 oligo using the BsmFI enzyme (Sample 1), no band corresponding to either the control oligo (undigested stem1 oligo), or the expected ligation product was present. The stem1 oligo was clearly digested by incubation with BsmFI, as evidenced by smaller fragments on the gel. In the lane corresponding to the bead sample which had been first cleaved with the BsmFI enzyme and subsequently ligated to the Alexa-488 labeled s1hp1 probe (Sample 2), there was a distinct band in the 488-imaging channel that ran at ~60 bases, consistent with the expected product size following ligation. This result demonstrated that the Alexa-488 labeled s1hp1 probe had been ligated onto the BsmFI digested stem1 oligo immobilized on the beads. Lastly, this 488-positive ligation band was lost in the lane containing the bead sample that had undergone a second digestion with BsmFI (Sample 3), indicating that the new BsmFI site introduced by ligation of the Alexa-488 labeled s1hp1 probe had been recognized by the enzyme, and that the stem1 oligo had been digested at the next correct encoded cleavage site. Thus, the enzyme had “ratcheted” down the stem1 oligo to the next designed sequence for ligation of a next correct labeled probe. These results are congruent with the bead imaging analysis.

FIG. 6 shows the OCLS method and workflow embodiment employed in this Example, and the results obtained from the method described. A ssDNA hairpin probe of the type shown in FIG. 3B (bottom), labeled with AF-488 was used. The labeled ssDNA hairpin probe contains an encoded RS, and was used for orthogonal ligation following cleavage of an OCLS barcode of the type depicted in FIG. 1B (FIG. 2, right side) containing multiple encoded CS. For the experiment outlined in FIG. 6 (schematic), an imaging step was performed in the 488 nm (emission) channel to determine bead signal intensities following the first OCLS barcode cleavage by the Type IIS RE, BsmFI. Following probe ligation and a subsequent wash step, the beads were imaged in the 488 nm channel for determining signal obtained from the label, and therefore the revealed overhang on the cleaved OCLS barcode. The same RS for the Type IIS RE that was used in the first cleavage reaction (BsmFI) was also encoded in the ligated probe, so following a subsequent wash to remove components from the previous reaction, this enzyme was added to the beads to digest the OCLS barcode for a second time. Following the second digestion and a wash step, an imaging step was performed in the 488 nm channel to determine signal obtained from the beads. Signal obtained across all three imaging steps in the 488 nm channel are plotted in the graph of FIG. 6, with relative fluorescence units (RFU) on the y-axis.

These experiments demonstrated ligation of the probe and subsequent cleavage of the probe were successful. Following the first cleavage by BsmFI (TIIS RE cut 1), beads contained nearly the same mean intensity value (<1,000 RFU) as they did after the second cleavage by BsmFI (TIIS RE cut 2), but following ligation of the ssDNA hairpin probe labeled with AF-488, the mean intensity value of beads was about 33,000 RFU. Ligation reactions containing a labeled, incorrect ssDNA hairpin probe (hairpin probe 3, containing an incorrect overhang for the OCLS barcode) did not create a ligation product (also determined by gel analysis).

It can be extrapolated from the result reported in this example that many different beads in a library that are encoded with different combinations of OCLS barcodes containing different CS can be decoded in highly parallel fashion using the OCLS workflow described here. This method involves ratcheting down the OCLS barcode by adding labeled orthogonal ligation probes containing encoded RS for use with Type IIS REs. In the illustrated embodiment, RS corresponding to Type IIS enzymes were used, and individual probes used in each cycle of decoding were labeled, ssDNA hairpin oligos containing specific overhangs for recognition of revealed overhangs on cleaved OCLS barcodes as shown in FIG. 6.

### Examples 2: OCLS Options, Using a Labeled, ssDNA Hairpin Probe

In methods to those described in Example 1 and elsewhere herein, optionally a different Type IIS RS is included in each subsequent probe that is used in each subsequent cycle, which beneficially can reduce aberrant cleavage across cycles. This approach will reduce the propensity for an enzyme that was used in a previous cycle to cleave an intact OCLS barcode that may have survived digestion (left un-cleaved) following a previous cleavage cycle.

Additionally, the use of labeled ssDNA hairpin oligos as the ligation probes enables a “clean-up” step following the ligation step, which can be performed before or after the imaging step. This clean-up step release on use of a 5′-exonuclease such as lambda exonuclease, which recognizes 5′-phosphorylated ends for digestion and removal of that strand. This optional clean-up step makes the digested barcodes incapable of interacting with any subsequent enzymes or probes used in future cycles (taking the out of the analysis from that point forward). The clean-up step removes any dsDNA that remains following a ligation step that was not 100% efficient, as ligated products (containing the labeled ssDNA hairpin oligos) can be designed such that they do not contain open 5′-phosphorylated ends.

### Example 3: Individual Segments and Chains of OCLS Barcodes Containing Standard RE Sites

In another embodiment, a set of standard orthogonal restriction enzymes can be used. In this case, OCLS barcodes can be purchased as fully constructed barcodes or as barcode segments from a vendor (such as IDT), containing one or more orthogonal recognition sites (RS). These barcodes can be built from segments as shown in FIG. 1A, and designed for specific cleavage at the RS by a site-specific restriction endonuclease enzyme (RE) as shown in FIG. 2 (left side), and can be used as illustrated in FIG. 4A. In this case, decoding can be achieved by immobilizing beads on a surface, such as a standard microscope slide or other surfaces/applications where feature identification and/or determining the locations of features is advantageous, followed by the introduction of one or more orthogonal RE (in a single reaction mixture), to bring the enzyme(s) into contact with one or more OCLS barcodes. As illustrated in FIG. 4A, following digestion of one or more OCLS barcodes by the RE enzyme(s), which will reveal specific overhangs on the OCLS barcodes, a wash step (for example, using 1× CutSmart buffer, New England Biolabs) would be performed to remove the RE enzyme(s), followed by a wash with 1×T4 DNA ligase buffer (for example, supplied by New England Biolabs with T4 DNA ligase enzyme; M0202). The 1×T4 DNA ligase buffer contains ATP and Mg²⁺, which are critical components for a successful ligation reaction. Following equilibration in ligase buffer, T4 DNA ligase and polynucleotide kinase (PNK; this enzyme will phosphorylate any un-phosphorylated 5′-ends of oligos, making them competent substrates for T4 DNA ligase) enzymes will be introduced in 1×T4 DNA ligase buffer containing labeled probes, which could for example, be accomplished in a flow cell equipped with microfluidics, or a microfluidic device under instrument control software for controlling both fluid flow rates and timing for enabling an in-line imaging step. This instrumental configuration is known to be somewhat standard in the art, and is used for methods and systems that require cycling of different solutions that are coordinated with imaging, such as next generation DNA sequencing by synthesis (SBS) workflows for example.

Here, each of the differentially-labeled probes would contain an overhang that is specific for one of the overhangs that would be generated on OCLS barcodes following the digestion step described above. Upon introduction of the enzymatic ligation solution containing the differentially-labeled probes, ligation between correctly matching overhangs (perfect base complementarity) of the OCLS barcode and one of the probes in the enzymatic ligation solution will occur. Following incubation with the enzymatic ligation solution, a wash step would be performed to remove the ligase and PNK enzymes for performing an imaging step. The imaging step would identify the specific overhanging sequence revealed on the OCLS barcodes following the described orthogonal digestion and ligation steps. Due to the fact that each orthogonal ligation probe will have a distinct label or combination of labels, distinguishing between different overhangs present on different OCLS barcodes can be enabled by an imaging step, or a step to otherwise specifically identify detectable probe labels associated with OCLS barcoded beads (as shown in FIG. 4B). Depending on the number of unique overhangs revealed on OCLS barcodes bound to individual beads or features following the digestion step, the ligation and imaging steps may be performed multiple times (as shown in FIG. 4C and FIG. 4D). This is because each orthogonal overhang generated on OCLS barcodes following the digestion step must be individually distinguished (individually read-out) by specific probes bearing labels or combinations of labels that correspond to the matching overhangs. It would also be possible to distinguish multiple different overhangs on single beads in this way during a single ligation event to create combinations of detectable labels on OCLS barcodes as shown in FIG. 4E. The OCLS method relies upon the generation of specific orthogonal overhangs on dsDNA OCLS barcodes (orthogonality is generated by RE digestion of OCLS barcodes), as well as the use of labeled probes containing both pre-determined labels and overhangs that correspond to (generate specific base complementarity with) the detected overhangs revealed on OCLS barcodes. In this way, many individual members of a library of beads or features can be distinguished over cycles, not unlike cycles performed in DNA sequencing workflows. In comparison, during NGS workflows, nucleotides containing both a pre-determined label and a base that corresponds to (generates specific base complementarity with) the next correct base in a primed nucleic acid template, are incorporated by a polymerase, and imaged for determining which base was incorporated in each cycle. Over multiple cycles, and in highly-parallel fashion, many beads or features can be sequenced by NGS or by OCLS.

### Example 4: Individual Segments and Chains of OCLS Barcodes Containing Type IIS RE Recognition Sites (RS) and/or Cleavage Sites (CS)

As described in Example 3, OCLS barcodes may contain one or more recognition site (RS) for cleavage by one or more standard RE, but in another embodiment, Type IIS RE enzymes can be used. Type IIS RE enzymes have RS that are distanced from their respective CS by a known number of bases. Type IIS RE enzymes will bind at their cognate RS, then these enzymes will cleave at their respective CS leaving a known number of overhanging bases. Specificity for Type IIS RE recognition of dsDNA occurs at the RS, but the CS can be any set of bases; these enzymes will cut at any designed CS, as long as it is the correct distance from the RS. Therefore, it is possible to generate OCLS barcodes with one or multiple CS, that contain no RS, or that contain one or more than one RS. As shown in FIG. 5A, experiments can be performed whereby two different barcodes are attached to individual beads that could be contained within a library of OCLS-barcoded beads. The two different OCLS barcodes on the bead can each contain a different combination of RS and CS for compatibility with two different (orthogonal) Type IIS enzymes. In this example, the beads could be immobilized on a surface for cycling as described in Example 2, and the two different Type IIS enzymes (BsmFI and BpuEI) could be introduced in a single reaction mixture consisting of a mutually compatible buffer such as NEB CutSmart buffer. As illustrated in FIG. 5A, digestion by these enzymes will remove a dsDNA cleavage product by cleaving at their respective CS, and not at the RS, which is different from standard REs (that is, REs that cut within their RS). Therefore, the CS can be designed in OCLS barcodes that are intended for use with Type IIS RE, as long as there is a RS positioned within the barcode at the correct distance for the specific enzyme being used, as shown in FIG. 5B. Each Type IIS enzyme substrate (OCLS barcodes) shown in FIG. 5B (BsmFI specific barcode shown above the BpuEI specific barcode on the bottom) will cleave at a CS that is a known number of bases from the RS, leaving a known number of bases in the resulting overhang on the barcode following digestion. Following cleavage by the Type IIS enzymes, a wash step (for example, using 1× CutSmart buffer from NEB) could be performed to remove the enzymes and cleaved products, followed by introduction of 1×T4 DNA ligase buffer for equilibration prior to a ligation step.

In this example, and as illustrated in FIG. 5C, two distinguishable overhangs on each feature would require two cycles of labeled probe ligation and imaging for identification of the two different overhangs produced following digestion. However, in this example, probes would not only contain distinguishable labels and corresponding overhangs as in Example 3, but would also contain an additional RS for a Type IIS RE (as demonstrated using ssDNA hairpin ligation probes in Example 1). Following ligation and imaging steps, the newly introduced RS will create a new “cassette” for the Type IIS enzyme (an RS positioned at an appropriate distance from the next CS in the OCLS barcode) by ligation of the probe to the correct OCLS barcode through matching overhangs. A wash step could be performed using 1× CutSmart buffer prior to introduction of Type IIS enzymes for the next cycle of digestion. By encoding a new Type IIS enzyme RS within the introduced labeled ligation probes, this process of “ratcheting” down the OCLS barcode, one designed CS per cycle, can be achieved. This was demonstrated in Example 1, using labeled a ssDNA hairpin oligo containing the BsmFI site, which was able to cleave the barcode a second time following the ligation step. Examples of oligos that can be used as barcodes in this way are included (TIIS stem1; SEQ ID NO: 1 and 2. TIIS stem2; SEQ ID NO: 12 and 13) and corresponding probe oligos that can be used for reading OCLS barcodes in this way are included (TIIS stem1 probes; SEQ ID NO: 3-11. TIIS stem2 probes; SEQ ID NO: 14-19).

The description provided herein is included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### CLOSING PARAGRAPHS

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials is individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A visual barcode, comprising:
at least two or more double-stranded DNA (dsDNA) oligonucleotide segments (cassettes) functionally linked linearly to each other, and each comprising within the sequence of the dsDNA segment:
a recognition site (RS) for a specific restriction endonuclease (RE),
a designed cleavage site (CS), or
both a RS and a CS;
and
an untethered end at one end of the visual barcode.

2. The visual barcode of claim 1, tethered to a solid substrate by a flexible linker attached at or near an end of the dsDNA of the visual barcode that is not the untethered end.

3. The visual barcode of claim 1, which does not comprise a visually detectable label.

4. The visual barcode of claim 1, wherein the RE is a Type IIS restriction endonuclease, and the CS does not overlap the corresponding RS.

5. The visual barcode of claim 1, wherein at least one of the dsDNA segments comprises a designed CS and the visual barcode comprises a RS specific for a Type IIS RE, positioned appropriately such that the cognate Type IIS RE can cut the designed CS based on its position relative to the RS.

6. The visual barcode of any one of claims 1-5, as illustrated in FIG. 1A or FIG. 1B or essentially as described herein.

7. A collection of visual barcodes of any one of claims 1-6, wherein the collection includes a plurality of visual barcodes each of which comprises a different set of dsDNA segments having different recognition sites (RSs) for specific restriction endonucleases (REs), designed cleavage sites (CSs), or both.

8. The collection of visual barcodes of claim 7, wherein at least two of the different visual barcodes are tethered to the same solid substrate.

9. An orthogonal cleavage-ligation sequencing (OCLS) barcode comprising two or more dsDNA segments (cassettes), each dsDNA segment containing a recognition site (RS) for a specific restriction endonuclease (RE), and one or more overlapping region(s) configured to permit ligation to flanking dsDNA segments to form a chain of segments, which chain of segments constitutes the OCLS barcode.

10. A visually detectable orthogonal ligation probe comprising:
a fully or partially double-stranded DNA oligonucleotide, having a 3′ or 5′ overhang of at least two nucleotides, the sequence of which includes:
a recognition site (RS) for a specific restriction endonuclease (RE),
a designed cleavage site (CS), or
both a RS and a CS;
and
covalently attached to the fully or partially dsDNA oligonucleotide, a visually detectable label.

11. The visually detectable orthogonal ligation probe of claim 10, wherein the fully or partially double-stranded DNA oligonucleotide comprises:
a linear double-stranded DNA oligonucleotide having a 5′ overhang;
a linear double-stranded DNA oligonucleotide having a 3′ overhang;
a hairpin stem-loop configured single-stranded DNA oligonucleotide having a 5′ overhang; or
a hairpin stem-loop configured single-stranded DNA oligonucleotide having a 3′ overhang.

12. The visually detectable orthogonal ligation probe of claim 10, wherein the visually detectable label is attached to the oligonucleotide by way of a flexible linker.

13. The visually detectable orthogonal ligation probe of claim 10, wherein the visually detectable label comprises one or more of a fluorescent label, a bioluminescent label, a chemiluminescent label, a chromophore, a quantum dot, a Raman label, a biotin moiety, or a radioactive isotope.

14. The visually detectable orthogonal ligation probe of claim 10, wherein the RE is a Type IIS restriction endonuclease, and RS is specific for that Type IIS RE.

15. The visually detectable orthogonal ligation probe of claim 10, comprising a RS specific for a Type IIS RE, positioned appropriately so the cognate Type IIS RE can cut a designed CS based on its position relative to the RS.

16. The visually detectable orthogonal ligation probe of any one of claims 10-15, as illustrated in FIG. 3A or FIG. 3B or essentially as described herein.

17. A collection of visually detectable orthogonal ligation probes of any one of claims 10-16, wherein the collection includes a plurality of visually detectable orthogonal ligation probes each of which comprises a different recognition site (RSs) for specific restriction endonucleases (REs), or both a different RS and a different cleavage site (CS).

18. The collection of visually detectable orthogonal ligation probes of claim 17, wherein at least two of the different detectable orthogonal ligation probes comprise visually distinguishable detectable labels.

19. The collection of visually detectable orthogonal ligation probes of claim 18, comprising a plurality of different probes each of which is configured such that cleavage of that probe with the RE produces an overhang having a sequence different from at least 5, at least 7, at least 10, at least 12, at least 15, or more than 15 other probes in the collection.

20. An orthogonal cleavage-ligation sequencing (OCLS) oligonucleotide pair, comprising:
a visual barcode, comprising:
at least two or more double-stranded DNA (dsDNA) oligonucleotide segments (cassettes) functionally linked linearly to each other, and each comprising within the sequence of the dsDNA segment:
a recognition site (RS) for a specific restriction endonuclease (RE),
a designed cleavage site (CS), or
both a RS and a CS;
and
an untethered end at one end of the visual barcode;
and
a visually detectable orthogonal ligation probe, comprising:
a fully or partially double-stranded DNA oligonucleotide, having a 3′ or 5′ overhang of at least two nucleotides, the sequence of which includes:
a recognition site (RS) for a specific restriction endonuclease (RE), or
both a RS and a cleavage site (CS); and
covalently attached to the fully or partially dsDNA oligonucleotide, a visually detectable label;
wherein cleavage of the RS or the CS in the visual barcode produces a single-stranded “sticky end” overhang having a sequence with full complementary to a sticky end (overhang) produced by cleavage of the RS in the visually detectable orthogonal ligation probe.

21. A set of OCLS oligonucleotides pair of claim 20, wherein each pair of visual barcode and visually detectable orthogonal ligation probe have a different fully complementary sequence overlap, and each visually detectable orthogonal ligation probe comprises a different visually distinguishable detectable label.

22. A method of encoding a visual barcode, comprising:
contacting a double-stranded DNA (dsDNA) oligonucleotide tethered at a first end to a solid support, which dsDNA oligonucleotide has a single-stranded overhang at a second, untethered end, with a first dsDNA segment having a first overhanging end compatible for binding to the single-stranded overhang of the tethered dsDNA oligonucleotide and a second overhanging end, and comprising within the sequence of the first dsDNA segment:
a recognition site (RS) for a specific restriction endonuclease (RE),
a designed cleavage site (CS), or
both a RS and a CS;
which contacting occurs under conditions sufficient to allow specific hybridization of the single-stranded overhang of the first dsDNA segment to the single-stranded overhang of the tethered dsDNA oligonucleotide;
ligating the first dsDNA segment to the tethered dsDNA oligonucleotide, to form a first captured barcode segment, which comprises the second overhanging end of the first dsDNA segment;
contacting the first captured barcode segment with a second dsDNA segment having a first overhanging end compatible for binding to the single-stranded overhang of the first captured barcode segment oligonucleotide and a second overhanging end, and comprising within the sequence of the second dsDNA segment:
a RS for a specific RE different from the RS/RE in the first dsDNA segment,
a designed CS different from the designed CS in the first dsDNA segment, or both;
and
ligating the second dsDNA segment to the first captured barcode segment, to form a second captured barcode segment, which comprises the second overhanging end of the second dsDNA segment, which first captured barcode segment and second captured barcode segment constitute the visual barcode.

23. The method of claim 22, further comprising repeating the contacting and ligating steps one or more additional times, each time attaching an additional dsDNA segment to the captured barcode segments, to form the visual barcode.

24. The method of claim 22 or claim 23, wherein at least one of the dsDNA segments comprises a designed CS and the visual barcode comprises a RS specific for a Type IIS RE, positioned appropriately so the cognate RE can cut the designed CS based on its position relative to the RS.

25. The method of any one of claims 22-24, as illustrated in FIG. 1A or FIG. 1B or as described herein.

26. The method of claim 24, wherein the visual barcodes are built using rounds of splitting and pooling using unlabeled DNA segments.

27. The method of claim 26, wherein one or more rounds of splitting and pooling comprise(s):
ligating one barcode cassette at a time onto a bead;
splitting the resultant beads into individual compartments, optionally wells of a plate;
ligating a different first compartment-specific barcode cassette onto the beads in each individual compartment, to yield a collection of beads containing different pairs of two barcode cassettes;
washing the beads containing two barcode cassettes;
pooling the beads containing two barcode cassettes;
splitting the pooled beads containing two barcode cassettes into individual compartments, optionally wells of a plate; and
repeating the ligating, washings, pooling, and splitting steps to increase diversity of the set of barcodes.

28. The method of any one of claims 22-27, wherein the visual barcode comprises a contiguous chain of dsDNA segments (cassettes), or the visual barcode comprises at least two separate cassettes attached directly and separately to the solid support.

29. The method of any one of claims 22-28, wherein the segments are co-encoded along with a next-generation-sequence (NGS) capture barcode.

30. A visual barcode made by the method of any one of claims 22-29.

31. A method of decoding a visual barcode, comprising:
contacting, in a milieu, at least one double-stranded DNA (dsDNA) orthogonal cleavage-ligation sequencing (OCLS) barcode comprising at least one restriction site (RS), with a restriction endonuclease (RE) that recognizes that RS, under conditions sufficient for the RE to cleave the dsDNA OCLS barcode, which RS/RE cleavage results in a single-stranded overhang to produce a partially single-stranded (ss) DNA-partially dsDNA OCLS barcode;
contacting the partially ssDNA-partially dsDNA OCLS barcode with at least one orthogonal ligation probe comprising a dsDNA oligonucleotide comprising an overhang at a first end and a visually detectable label, under conditions sufficient for the overhang of the orthogonal ligation probe to bind by base pair-mediated hydrogen bonding to the overhang on the partially ssDNA-partially dsDNA OCLS barcode if the sequence of the overhang of the orthogonal ligation probe is the reverse complement of the sequence of the overhang of the partially ssDNA-partially dsDNA OCLS barcode;
if base pair-mediated binding occurs, ligating the orthogonal ligation probe to the partially single-stranded (ss) DNA-partially dsDNA OCLS barcode to produce a captured probe; and
detecting presence, absence, and/or quantity of captured probe by imaging the visually detectable label.

32. The method of claim 31, further comprising repeating the contacting/cleavage, contacting/base pair-mediated binding, ligating, and detecting steps cycle one or more times, where each additional cycle involves a cleavage of the dsDNA OCLS barcode at a different cleavage site (CS), base pair-mediated binding of a different orthogonal ligation probe, and/or detection of the presence, absence, and/or quantity of a different labeled captured probe.

33. The method of claim 31, wherein one or more of:
the dsDNA OCLS barcode is attached to a bead or other solid surface;
a plurality of different dsDNA OCLS barcodes are attached to a single bead or single address on another solid surface;
the dsDNA OCLS barcode comprises more than one non-overlapping RSs;
at least one RS in the dsDNA OCLS barcode is recognized by a Type IIS RE, and cleavage occurs at a predetermined location outside of the RS;
at least one RS in the dsDNA OCLS barcode is recognized by a RE that cleaves within the RS; or
the visually detectable label comprises at least one of a fluorescent label, a bioluminescent label, a chemiluminescent label, a chromophore, a quantum dot, a Raman label, or a radioactive isotope.

34. The method of claim 32, wherein one or more of:
the orthogonal ligation probe comprises a single stranded DNA oligonucleotide having a stem-loop hairpin structure, wherein the overhang is at the end of the stem of the hairpin.

35. The method of claim 34, after ligating, further comprising contacting the milieu comprising the captured probe with a 5′-exonuclease.

36. The method of claim 32, comprising one or more of:
contacting the at least one dsDNA OCLS barcode sequentially with two or more REs that each recognize a different, non-overlapping RS within the dsDNA OCLS under conditions sufficient for each RE to cleave the dsDNA OCLS barcode, each of which RS/RE cleavage results in a single-stranded overhang to produce a partially single-stranded (ss) DNA-partially dsDNA OCLS barcode;
the ligating comprises chemical ligation; or
the ligating comprises enzyme-mediated ligation.

37. The method of claim 31, comprising:
contacting two or more dsDNA OCLS barcodes, each comprising at least one RS, with a RE that recognizes that RS under conditions sufficient for the RE to cleave the dsDNA OCLS barcode, which RS/RE cleavage results in a single-stranded overhang to produce a partially single-stranded (ss) DNA-partially dsDNA OCLS barcode, wherein the RS/RE is different for each dsDNA OCLS barcode.

38. The method of claim 37, comprising contacting the two or more dsDNA OCLS barcodes with two or more orthogonal REs.

39. The method of claim 38, wherein the contacting with two or more orthogonal REs is simultaneous or sequential.

40. The method of claim 39, wherein following contacting with the one or more orthogonal REs, differentially-labeled probes are ligated using specific orthogonal reactions.

41. The method of claim 40, wherein the specific orthogonal ligation of differentially-labeled probes enables visual distinguishing of the barcodes at each feature in an array of visual barcodes.

42. The method of claim 31, wherein ligating the orthogonal ligation probe to the partially ssDNA-partially dsDNA OCLS barcode adds a new RS to the resultant a captured probe.

43. A method of decoding visual barcodes using orthogonal cleavage-ligation sequencing (OCLS) essentially as described herein.

44. An improved system for molecular barcoding, comprising repeated cycles of labeling, orthogonal cleavage, ligation, and imaging in order to identify individual features, wherein the orthogonal cleavage comprises cleaving a double-stranded DNA barcode with a Type IIS Restriction Endonuclease.

45. The system of claim 44, wherein two or more labeling and orthogonal probe ligation identification cycles occur in series, or occur concurrently.

46. Orthogonal cleavage-ligation sequencing (OCLS) system, as illustrated in FIGS. 5A-5E.


SEQUENCE LISTING (19 sequences)

SEQ ID NO: 1 - DNA, 60 bp, synthetic construct
  source 1..60: mol_type = other DNA
  misc_feature 1: note = 5 prime flexible 6 carbon atom linker bearing a reactive primary amine linked to base
ttttttattatattacgctaatagcattatatcatgttaacgcaatgtcccatatcgcta

SEQ ID NO: 2 - DNA, 54 bp, synthetic construct
  source 1..54: mol_type = other DNA
tagcgatatgggacattgcgttaacatgatataatgctattagcgtaatataat

SEQ ID NO: 3 - DNA, 47 bp, synthetic construct
  source 1..47: mol_type = other DNA
  modified_base 1: mod_base = OTHER; note = 5 prime phosphorylation
  modified_base 26: mod_base = OTHER; note = internal 6-carbon atom amine-modified thymine
catgcgtcccgcgtcggatacgtttttttcgtatccgacgcgggacg

SEQ ID NO: 4 - DNA, 47 bp, synthetic construct
  source 1..47: mol_type = other DNA
  modified_base 1: mod_base = OTHER; note = 5 prime phosphorylation
  modified_base 26: mod_base = OTHER; note = internal 6-carbon atom amine-modified thymine
atgccgtcccgcgtcggatacgtttttttcgtatccgacgcgggacg

SEQ ID NO: 5 - DNA, 47 bp, synthetic construct
  source 1..47: mol_type = other DNA
  modified_base 1: mod_base = OTHER; note = 5 prime phosphorylation
  modified_base 26: mod_base = OTHER; note = internal 6-carbon atom amine-modified thymine
gcgtcgtcccgcgtcggatacgtttttttcgtatccgacgcgggacg

SEQ ID NO: 6 - DNA, 21 bp, synthetic construct
  source 1..21: mol_type = other DNA
  misc_feature 1: note = 5 prime flexible 6 carbon atom linker bearing a reactive primary amine linked to base
tttcgtatccgacgcgggacg

SEQ ID NO: 7 - DNA, 25 bp, synthetic construct
  source 1..25: mol_type = other DNA
  modified_base 1: mod_base = OTHER; note = 5 prime phosphorylation
catgcgtcccgcgtcggatacgaaa

SEQ ID NO: 8 - DNA, 21 bp, synthetic construct
  source 1..21: mol_type = other DNA
  misc_feature 1: note = 5 prime flexible 6 carbon atom linker bearing a reactive primary amine linked to base
tttcgtatccgacgcgggacg

SEQ ID NO: 9 - DNA, 25 bp, synthetic construct
  source 1..25: mol_type = other DNA
  modified_base 1: mod_base = OTHER; note = 5 prime phosphorylation
atgccgtcccgcgtcggatacgaaa

SEQ ID NO: 10 - DNA, 21 bp, synthetic construct
  source 1..21: mol_type = other DNA
  misc_feature 1: note = 5 prime flexible 6 carbon atom linker bearing a reactive primary amine linked to base
tttcgtatccgacgcgggacg

SEQ ID NO: 11 - DNA, 25 bp, synthetic construct
  source 1..25: mol_type = other DNA
  modified_base 1: mod_base = OTHER; note = 5 prime phosphorylation
gcgtcgtcccgcgtcggatacgaaa

SEQ ID NO: 12 - DNA, 60 bp, synthetic construct
  source 1..60: mol_type = other DNA
  misc_feature 1: note = 5 prime flexible 6 carbon atom linker bearing a reactive primary amine linked to base
ttttttattatattaactaatatgtatatagtgttaacagtcaatctcaagatatcgcta

SEQ ID NO: 13 - DNA, 54 bp, synthetic construct
  source 1..54: mol_type = other DNA
tagcgatatcttgagattgactgttaacactatatacatattagttaatataat

SEQ ID NO: 14 - DNA, 31 bp, synthetic construct
  source 1..31: mol_type = other DNA
  misc_feature 1: note = 5 prime flexible 6 carbon atom linker bearing a reactive primary amine linked to base
tttcgtatccgacgcgcttgaggtacgacct

SEQ ID NO: 15 - DNA, 29 bp, synthetic construct
  source 1..29: mol_type = other DNA
  modified_base 1: mod_base = OTHER; note = 5 prime phosphorylation
gtcgtacctcaagcgcgtcggatacgaaa

SEQ ID NO: 16 - DNA, 31 bp, synthetic construct
  source 1..31: mol_type = other DNA
  misc_feature 1: note = 5 prime flexible 6 carbon atom linker bearing a reactive primary amine linked to base
tttcgtatccgacgcgcttgaggtacgacca

SEQ ID NO: 17 - DNA, 29 bp, synthetic construct
  source 1..29: mol_type = other DNA
  modified_base 1: mod_base = OTHER; note = 5 prime phosphorylation
gtcgtacctcaagcgcgtcggatacgaaa

SEQ ID NO: 18 - DNA, 31 bp, synthetic construct
  source 1..31: mol_type = other DNA
  misc_feature 1: note = 5 prime flexible 6 carbon atom linker bearing a reactive primary amine linked to base
tttcgtatccgacgcgcttgaggtacgacgt

SEQ ID NO: 19 - DNA, 29 bp, synthetic construct
  source 1..29: mol_type = other DNA
  modified_base 1: mod_base = OTHER; note = 5 prime phosphorylation
gtcgtacctcaagcgcgtcggatacgaaa